(19) United States
(12) Patent Application Publication
(10) Pub. No.: US 2013/0253187 A1
(43) Pub. Date: Sep. 26, 2013

(54) Process for Determining the Suitability for Distribution of a Batch of Thiophene-2-Carboxamide Derivative

(71) Applicants: Bernardino Mangion, Pembroke (MT); Ernesto Duran Lopez, Castellbisbal- Barcelona (ES)

(72) Inventors: Bernardino Mangion, Pembroke (MT); Ernesto Duran Lopez, Castellbisbal- Barcelona (ES)

(73) Assignee: Medichem, S.A., Barcelona (ES)

(21) Appl. No.: 13/823,256

(22) PCT Filed: Sep. 14, 2010
(86) PCT No.: PCT/EP11/65925

§ 371 Date: May 30, 2013

Related U.S. Application Data
(60) Provisional application No. 61/382,669, filed on Sep. 14, 2010.

(51) Int. Cl. C07D 413/14; G01N 33/15

(52) U.S. Cl. CPC C07D 413/14; G01N 33/15
USPC 544/82; 544/137; 73/61.52

(57) ABSTRACT

The present invention relates to a process for determining the suitability for distribution of a batch of rivaroxaban or of a pharmaceutical composition thereof. In particular, it also relates to two impurities of rivaroxaban, to their use as reference markers to determine the purity of a sample of rivaroxaban or a composition thereof, to analytical methods for determining the purity of a sample of rivaroxaban or a composition thereof and to a process of preparing rivaroxaban or pharmaceutical compositions thereof which are free or substantially free of such impurities.

### FIELD OF THE INVENTION

The present invention relates to a process for determining the suitability for distribution of a batch of rivaroxaban or of a pharmaceutical composition thereof. In particular, it also relates to two impurities of rivaroxaban, to their use as reference markers to determine the purity of a sample of rivaroxaban or a composition thereof, to analytical methods for determining the purity of a sample of rivaroxaban or a composition thereof and to a process of preparing rivaroxaban or pharmaceutical compositions thereof which are free or substantially free of such impurities.

### BACKGROUND OF THE INVENTION

Rivaroxaban (compound I) is the international commonly accepted name for (S)-5-chloro-N-{[2-oxo-3-[4-(3-oxomorpholin-4-yl)phenyl]-1,3-oxazolidin-5-yl)methyl}thiophene-2-carboxamide and has an empirical formula of C₁₉H₁₈N₃O₅SCl and a molecular weight of 435.88 g/mol.

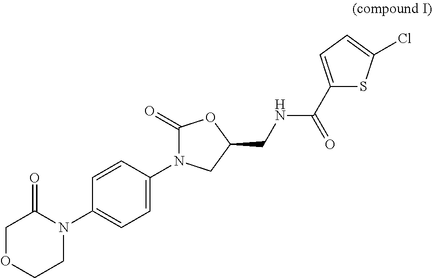 [SMILES: O=C(NC[C@H]1CN(c2ccc(N3CCOCC3=O)cc2)C(=O)O1)c1ccc(Cl)s1]

Rivaroxaban acts as inhibitor of clotting factor Xa and is indicated for the prevention of venous thromboembolism (VTE) in adult patients undergoing elective hip or knee replacement surgery. Rivaroxaban is marketed in the form of film-coated tablets under the trademark XARELTO™.

The synthesis of rivaroxaban was first described in Example 44 of U.S. Pat. No. 7,576,111 (“the '111 patent”, see Scheme 1). In this example, (R)-2-(2-hydroxy-3-{[4-(3-oxomorpholin-4-yl)phenyl]amino}propyl)-1H-isoindole-1,3(2H)-dione (compound II) is obtained by reaction between 4-(4-aminophenyl)-3-morpholinone and (S)-2-[2-oxiranylmethyl]-1H-isoindole-1,3(2H)-dione in an ethanol/water mixture, and isolated after recrystallization with an HPLC purity of 100%. Subsequently, compound (II) is reacted with N,N′-carbonyldiimidazole (CDI) in the presence of a catalytic amount of dimethylaminopyridine, using tetrahydrofuran as solvent, to obtain (S)-2-({2-oxo-3-[4-(3-oxomorpholin-4-yl)phenyl]-1,3-oxazolidin-5-yl}methyl)-1H-isoindole-1,3(2H)-dione (compound III) which is isolated with an HPLC purity of 100% after concentration under reduced pressure followed by flash chromatography purification. Finally, the phthalimide protective group of compound (III) is removed by reaction with methylamine to give (S)-4-{4-[5-(aminomethyl)-2-oxo-1,3-oxazolidin-3-yl]phenyl}morpholin-3-one (compound IV), which is obtained by concentration of the reaction mixture and employed directly without further purification in the reaction with 5-chlorothiophene-2-carbonyl chloride using pyridine as solvent to afford rivaroxaban, which is isolated with an HPLC purity of 100% after flash chromatography purification.

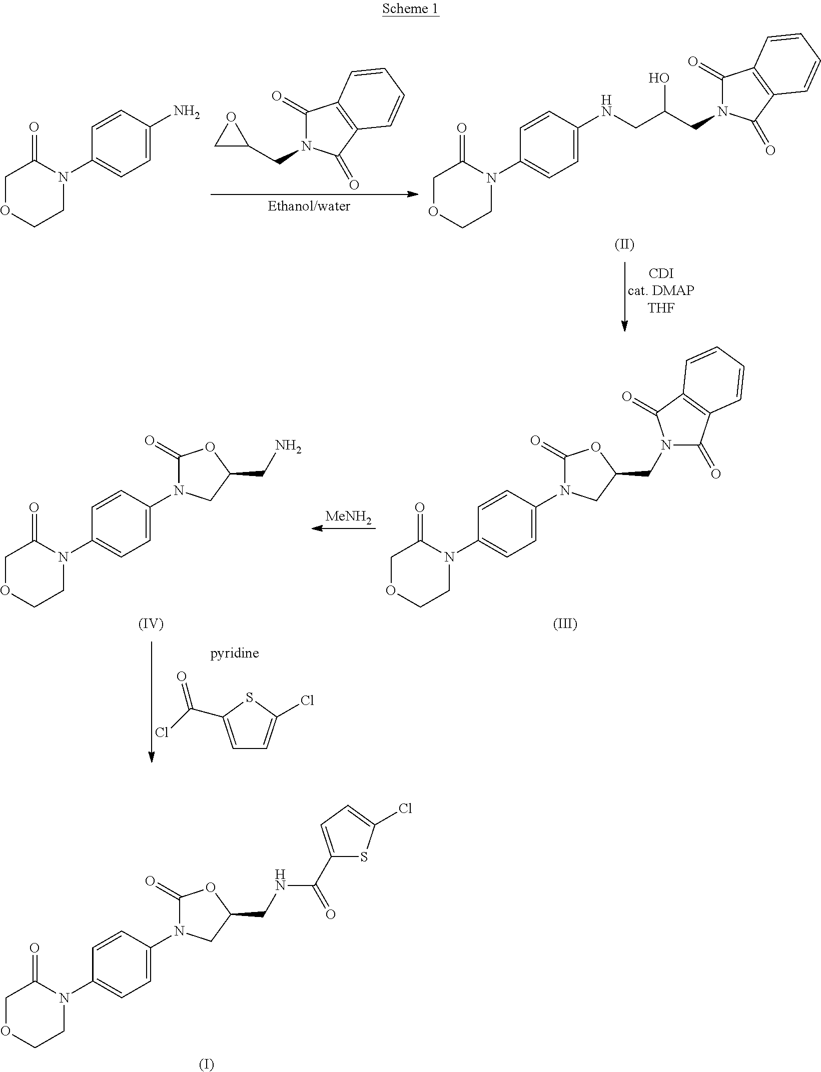

Although this method describes the isolation of rivaroxaban with high purity, the need of chromatographic purification of both compounds (III) and (I) makes this process not suitable for the synthesis of rivaroxaban at industrial scale. Furthermore, U.S. Pat. No. 7,351,823 (“the '823 patent”) describes that the process disclosed in Example 44 of the '111 patent exhibits various disadvantages in the reaction management which has particularly unfavorable effects for preparation of the compound of the formula (I) at an industrial scale.

The '823 patent discloses some modifications of the process described in the '111 patent to overcome these unfavorable disadvantages. Particularly, the isolation of compound (IV) as solid hydrochloride in pure form is described to make improved reaction management possible in the subsequent reaction with 5-chlorothiophene-2-carbonyl chloride, with unwanted side reactions being avoided and a purer product being obtained, so that the elaborate chromatographic purification of rivaroxaban (compound I) can be avoided. Additionally, compound (II) is described to react with N,N′-carbonyldiimidazole (CDI) in the absence of catalyst and using N-methyl-2-pyrrolidone (NMP) or toluene as solvent, which allows the isolation of compound (III) by filtration instead of by elaborate chromatographic purification. Finally, the reaction between compound (IV) (in form of its hydrochloride salt) and 5-chlorothiophene-2-carbonyl chloride is carried out in a solvent selected from the group of ether, alcohol, ketone and water or in a mixture thereof with use of an inorganic base, thus avoiding the use of carcinogenic pyridine (which is used as solvent and base in the process of the '111 patent) and therefore its presence as impurity in final rivaroxaban. Particularly mentioned examples of preferred inorganic bases are sodium hydroxide, sodium carbonate or sodium bicarbonate, especially sodium carbonate.

The '823 patent, however, does not disclose the chemical purity of the rivaroxaban obtained by the modified process. The experimental example (d, 3^{rd} step) describes a melting point of 230° C. for rivaroxaban after recrystallization from acetic acid, filtration, washing with acetic acid and water and drying. The reported melting point is lower than the melting point of rivaroxaban with 100% chemical purity described in the '111 patent, i.e. 232-233° C., and which corresponds to crystalline form I.

The authors of the present invention have found that the rivaroxaban obtained by the modified process disclosed in the '823 patent contains high amounts of specific impurities, and therefore the process is not suitable for the preparation of compound (I) on an industrial scale. One significant undesired by-product found in the rivaroxaban obtained by the modified process disclosed in the example (d, 2^{nd} step) of the '823 patent is the N,N-bis[{(5S)-2-oxo-3-[4-(3-oxomorpholin-4-yl)phenyl]-1,3-oxazolidin-5-yl}methyl]urea, hereinafter referred as Compound A, which has not been previously described in the literature:

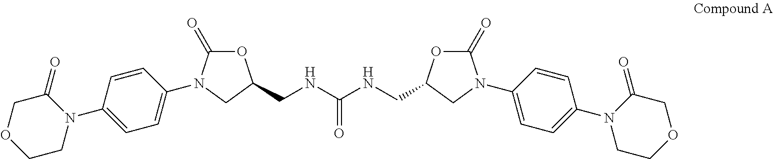 [SMILES: O=C(NC[C@H]1CN(c2ccc(N3CCOCC3=O)cc2)C(=O)O1)NC[C@H]1CN(c2ccc(N3CCOCC3=O)cc2)C(=O)O1]

This Compound A is not completely removed after the recrystallization process from acetic acid disclosed in the '823 patent. Furthermore the acetic acid is not a suitable industrially applicable solvent, since it implies some drawbacks as such related, for example, to its odor.

U.S. Patent application No. 20100152189 reports processes which comprise contacting rivaroxaban form I with several solvents in order to obtain other polymorphic forms of rivaroxaban, such as amorphous form, form II, form III, a hydrate, a N-methyl-2-pyrrolidone (NMP) solvate and an inclusion compound with tetrahydrofuran (THF).

Example 2.1 in U.S. Patent application No. 20100152189 discloses the reaction between an oxamine hydrochloride and 5-chlorothiophene-2-carbonyl chloride in the presence of triethylamine and N-methyl-2-pyrrolidone (NMP) as solvent to give, allegedly, rivaroxaban modification II. Again these processes involve the use of solvents showing particular disadvantages for an industrial application like NMP which may cause harm to the unborn child or THF whose flash point is −14.5° C.

Regulatory authorities worldwide require that drug manufacturers control the levels of the impurities in the final drug compound obtained by the manufacturing process and ensure that the impurities are present in the lowest possible levels.

For purposes of the present invention, any of the residual solvents present in rivaroxaban are also considered as impurities.

In view of the foregoing, there is a need to provide improved processes for preparing rivaroxaban which are suitable for industrial implementation and which produce rivaroxaban substantially free of impurities and in high yields.

### DETAILED DESCRIPTION OF THE INVENTION

It is an object of the present invention to provide an inexpensive, environmentally friendly, simple and economical process suitable for industrial application for the preparation of rivaroxaban having desired quality properties.

The first aspect of the present invention is a process for preparing (S)-5-chloro-N-{[2-oxo-3-[4-(3-oxomorpholin-4-yl)phenyl]-1,3-oxazolidin-5-yl)methyl}thiophene-2-carboxamide (rivaroxaban/Compound I), comprising reacting (S)-4-{4-[5-(aminomethyl)-2-oxo-1,3-oxazolidin-3-yl]phenyl}morpholin-3-one (compound IV) or salts thereof with 5-chlorothiophene-2-carbonyl chloride in the presence of an organic base with a pKₐ higher than 5.3, or mixtures thereof. Particularly preferred organic bases are organic bases with a pKₐ higher than 8.5, more preferably organic bases with a pKₐ higher than 10.0. Furthermore a preferred organic base is selected from the group of tertiary amines and amidine bases having a pKₐ higher than 5.3, more preferably tertiary amines selected from the group of N,N-diisopropylethylamine (DIPEA), trimethylamine, tripropylamine, N-methylpiperidine, N,N-dimethylaminopyridine (DMAP), N-methylpyrrolidine and 1,4-diazabicyclo[2.2.2]octane (DABCO), or amidine bases selected from the group of 1,5-diazabicyclo[4.3.0]non-5-ene (DBN) and 1,8-diazabicyclo[5.4.0]undec-7-ene (DBU), or mixtures thereof, and even more preferably N,N-diisopropylethylamine (DIPEA).

The use of organic bases with a pKₐ higher than 5.3, in particular N,N-diisopropylethylamine (DIPEA), allows the use of lower amounts of the same to carry out the reaction between compound (IV) and 5-chlorothiophene-2-carbonyl chloride with excellent conversions. Thus, it is possible to design cheaper processes which involve easier isolation steps by minimizing the presence of organic base residues in the final rivaroxaban. Using organic bases with a pKₐ lower than 5.3, for example pyridine in the '111 patent, makes necessary to use large amounts of the same and therefore making more difficult the isolation of a rivaroxaban with acceptable amounts of the organic bases residues.

The pKₐ is a measurement of the strength of an acid. The lower the pKₐ, the stronger the acidity. The higher the pKₐ, the weaker the acid. The pK_{b} is a related measurement and is a measurement of the strength of a base; the lower the pK_{b}, the stronger the base and the higher the pK_{b}, the weaker the base. Although it is not strictly accurate, often the pKₐ of a base's conjugated acid is provided as the pKₐ of the base. In this application the term pKₐ of a base is used to designate the pKₐ of the base's conjugated acid. For example, one would give a value of 5.25 for the pKₐ of pyridine meaning that the pKₐ of the pyridinium ion C₅H₆N⁺ is 5.25. In this application the pKₐ values refer to the pKₐ in water as determined at room temperature and atmospheric pressure.

On the other hand, the use of an organic base instead of an inorganic base as it is reported in the '823 patent gives more versatility to the reaction between compound (IV) and 5-chlorothiophene-2-carbonyl chloride. Due to the high solubility of organic bases in both organic solvents and in water, the reaction between compound (IV) and 5-chlorothiophene-2-carbonyl chloride can take place in both organic media and in aqueous media as well as in biphasic systems, where the fact that the base is dissolved in both phases provides a more effective reaction.

Furthermore, the authors of the present invention have observed that when N,N-diisopropylethylamine is used as a base the yield of the reaction between compound (IV) and 5-chlorothiophene-2-carbonyl chloride is around 97.9%. That is at least around 3% higher than the yield obtained following the process of the '823 patent (a 94.7% is given in example d, 2^{nd} step of the '823 patent and a 93.1% is found by the authors of the present invention). A yield increase of around 3%, which could be considered as irrelevant at a laboratory scale, has a significant impact in the implementation of a process at industrial scale from both environmental and economic points of view.

The authors of the present invention have surprisingly found that the use of triethylamine in the reaction between (S)-4-{4-[5-(aminomethyl)-2-oxo-1,3-oxazolidin-3-yl]phenyl}morpholin-3-one (compound IV) or salts thereof and 5-chlorothiophene-2-carbonyl chloride gives rivaroxaban with substantially lower yields and purities than those obtained with the use of N,N-diisopropylethylamine.

Compound (IV) can be used in form of the free base (either in solution from a previous synthetic step or in isolated form) or in form of a salt thereof. Non-limiting examples of salts of compound (IV) are hydrochloride, hydrobromide, sulfate, mesylate, tartrate, phosphate, citrate, fumarate, tosylate, benzoate, mandelate, succinate, oxalate, camphorsulfonate and maleate, preferably hydrochloride and sulfate, and more preferably the sulfate salt (2:1) of compound (IV).

The reaction between compound (IV) and 5-chlorothiophene-2-carbonyl chloride takes place preferably in the presence of a solvent. Non-limiting examples of suitable solvents which can be used are: ethers such as tetrahydrofuran, dioxane, diisopropylether, diethylether, 2-methyltetrahydrofuran, cyclopentyl methyl ether or methyl tert-butyl ether; ketones such as methyl ethyl ketone, methyl isobutyl ketone or acetone; halogenated solvents such as dichloromethane, chloroform, tetrachloromethane, dichloroethane, chlorobenzene or dichlorobenzene; polar aprotic solvents such as N,N-dimethylformamide, acetonitrile, N,N-dimethylacetamide, N-methyl-2-pyrrolidone or dimethylsulfoxide; hydrocarbon aliphatic solvents such as methylcyclohexane, cyclohexane, heptane or hexane; hydrocarbon aromatic solvents such as toluene, benzene, o-xylene, m-xylene or p-xylene or water or mixtures of two or more of the solvents listed. Particularly preferred solvents are ethers, ketones, water or mixtures thereof. More preferred solvents are ketones or mixtures comprising ketones and water, and even more preferred solvents are mixtures comprising acetone and water.

Thus, a preferred embodiment of the present invention provides a process for preparing rivaroxaban (compound I) comprising reacting (S)-4-{4-[5-(aminomethyl)-2-oxo-1,3-oxazolidin-3-yl]phenyl}morpholin-3-one (compound IV), preferably in form of a salt, with 5-chlorothiophene-2-carbonyl chloride in a solvent, preferably comprising ethers, ketones, water or mixtures thereof, in the presence of an organic base having a pKₐ higher than 5.3, preferably N,N-diisopropylethylamine (DIPEA).

Particularly preferred conditions for the process of the present invention are summarized in Scheme 2.

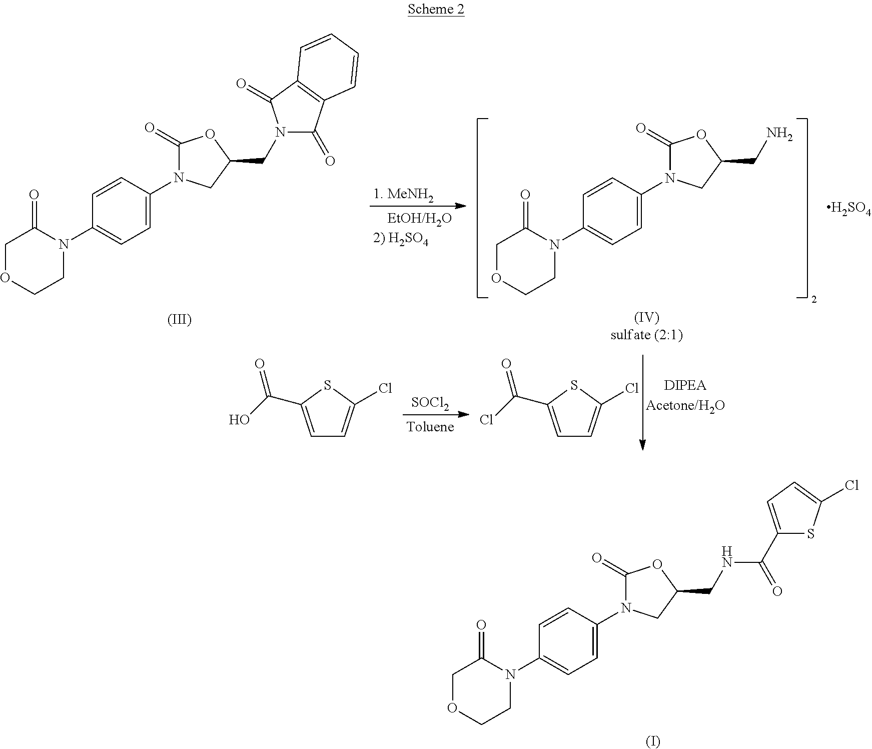

Rivaroxaban obtained according to the process of the present invention preferably undergoes a further recrystallization.

The term of “recrystallization” as used herein comprises precipitation by dissolving in a solvent or mixture of solvents and adding an anti-solvent or a mixture of anti-solvents, precipitation by dissolving in a solvent or mixture of solvents and cooling, or precipitation by dissolving in a solvent or mixture of solvents and seeding.

Preferred solvents and/or antisolvents used in the recrystallization according to the present invention are acetic acid, N,N-dimethylformamide, N,N-dimethylacetamide, N-methyl-2-pyrrolidone, dimethylsulfoxide or acetonitrile or mixtures thereof.

The term “anti-solvent” is used in the present invention to denote an organic solvent that functions by reducing the solubility of rivaroxaban in another solvent (the primary solvent) without affecting rivaroxaban from the chemical standpoint.

In a particularly preferred embodiment of the present invention rivaroxaban is recrystallized in a mixture of solvents comprising dimethylsulfoxide and acetonitrile, wherein the dimethylsulfoxide/acetonitrile volume/volume ratio is from about 2/1 to about 1/5, preferably from about 1/1 to about 2/5, more preferably about 3/5.

The process of recrystallization according to the present invention can include only one recrystallization step or more than one consecutive recrystallization steps.

The process of recrystallization of rivaroxaban according to the present invention preferably comprises the following steps: (a) providing a rivaroxaban solution in a solvent or a mixture of solvents at a temperature between 60° C. and 130° C., preferably between 80° C. and 110° C., more preferably between 90° C. and 100° C., and even more preferably at 95±2° C.; (b) cooling down the solution obtained in step (a) to a temperature between −5° C. and 30° C., preferably between 0° C. and 20° C., more preferably between 5° C. and 10° C. over a period of time between 20 minutes and 24 hours; (c) optionally, maintaining the suspension obtained in step (b) at a temperature between −5° C. and 30° C., preferably between 0° C. and 20° C., more preferably between 5° C. and 10° C. under stirring over a period of time between 5 minutes and 24 hours; and (d) isolating rivaroxaban from the suspension obtained in steps (b) or (c).

Optionally the process of recrystallization of rivaroxaban according to the present invention further comprises a step of seeding the solution of rivaroxaban with rivaroxaban, preferably in crystalline form I as described in WO2007039132A2, in order to better control and/or facilitate the precipitation of rivaroxaban.

Thus, optionally the process of recrystallization of rivaroxaban according to the present invention comprises the following steps: (a) providing a rivaroxaban solution in a solvent or a mixture of solvents at a temperature between 60° C. and 130° C., preferably between 80° C. and 110° C., more preferably between 90° C. and 100° C., and even more preferably at 95±2° C.; (b) seeding with rivaroxaban the solution obtained in step (a) at a temperature between 60° C. and 130° C., preferably between 80° C. and 100° C., more preferably between 85° C. and 95° C., and even more preferably at 88±2° C.; (c) optionally, maintaining the mixture obtained in step (b) at a temperature between 60° C. and 130° C., preferably between 70° C. and 100° C., more preferably between 80° C. and 90° C., and even more preferably at 85±2° C. over a period of time between 1 hour and 24 hours; (d) cooling down the mixture obtained in step (b) or (c) to a temperature between −5° C. and 30° C., preferably between 0° C. and 20° C., more preferably between 5° C. and 10° C. over a period of time between 20 minutes and 24 hours; (e) optionally, maintaining the suspension obtained in step (d) at a temperature between −5° C. and 30° C., preferably between 0° C. and 20° C., more preferably between 5° C. and 10° C. under stirring over a period of time between 5 minutes and 24 hours; and (f) isolating rivaroxaban from the suspension obtained in steps (d) or (e).

The term “seeding with rivaroxaban” when used herein refers to the addition of rivaroxaban crystals onto a solution of rivaroxaban to control and/or facilitate the precipitation of rivaroxaban.

Preferably the process may optionally further comprise any stage of decoloration and/or removal of insoluble particles by means of filtering a solution of rivaroxaban in a solvent or mixture of solvents, optionally also containing a decolorizing agent such as silica gel or charcoal, preferably charcoal.

Optionally rivaroxaban obtained according to the process of the present invention can undergo a further slurrying.

The term “slurrying” as used herein refers to combine rivaroxaban with a solvent or mixture of solvents so that at any time the rivaroxaban stays totally or partially suspended in the solvent or mixture of solvents.

Preferred solvents used for the slurrying process according to the present invention are alcohols such as methanol, ethanol, isopropanol, n-propanol, n-butanol, isobutanol, sec-butanol or tert-butanol; ketones such as acetone, methyl ethyl ketone or methyl isobutyl ketone; ethers such as tetrahydrofuran, dioxane, diethylether, diisopropylether, cyclopentyl methyl ether, 2-methyltetrahydrofuran or methyl tert-butyl ether; esters such as ethyl acetate, methyl acetate, isopropyl acetate, n-propyl acetate, n-butyl acetate, isobutyl acetate, sec-butyl acetate or tert-butyl acetate; hydrocarbon aliphatic solvents such as cyclohexane, methylcyclohexane, heptane or hexane; halogenated solvents such as dichloromethane, chloroform, tetrachloromethane, dichloroethane, chlorobenzene or dichlorobenzene; hydrocarbon aromatic solvents such as toluene, benzene, o-xylene, m-xylene or p-xylene; carboxylic acids such as acetic acid or formic acid; amides such as N,N-dimethylformamide or N,N-dimethylacetamide or N-methyl-2-pyrrolidone; sulfoxides such as dimethylsulfoxide; nitriles such as acetonitrile or water, or mixtures of two or more of the solvents listed. Preferred solvents used for the slurrying process according to the present invention are ketones, more preferably acetone; sulfoxides such as dimethylsulfoxide; nitriles such as acetonitrile or mixtures of two or more of the solvents listed, e.g. mixtures of dimethylsulfoxide and acetonitrile.

Alternatively rivaroxaban obtained according to the process of the present invention can undergo a further recrystallization process as disclosed herein followed by a slurrying process as disclosed herein.

The authors of the present invention have also found that recrystallization and/or slurrying processes according to the present invention yield a rivaroxaban substantially free of occluded residual organic solvents.

The term “occluded residual solvent” refers to the solvent molecules which stay trapped within the crystalline structure of rivaroxaban and which are not removed after conventional drying processes.

The term “substantially free” of residual organic solvents as used herein means that rivaroxaban obtained according to the recrystallization and/or slurrying according to the present invention contains, after drying, less than about 5000 ppm of any individual residual organic solvent, preferably less than about 1000 ppm of any individual residual organic solvent. Particularly, rivaroxaban obtained according to the recrystallization or the recrystallization followed by the slurrying of the present invention contains, after drying, less than about 5000 ppm of any individual residual class 3 organic solvent, preferably less than about 1000 ppm of any individual residual class 3 organic solvent, and less than about 5000 ppm of any individual residual class 2 organic solvent, preferably less than about 1000 ppm of any individual residual class 2 organic solvent, more preferably less than about 700 ppm of any individual residual class 2 organic solvent, and even more preferably less than about 400 ppm of any individual residual class 2 organic solvent. Class 2 and class 3 organic solvents are disclosed in the Guideline for Residual Solvents Q3C(R5) of the International Conference on Harmonisation of Technical Requirements for Registration of Pharmaceuticals for Human Use (ICH). Concretely, class 2 organic solvents are solvents of the group comprising acetonitrile, chlorobenzene, chloroform, cumene, cyclohexane, 1,2-dichloroethane, dichloromethane, 1,2-dimethoxyethane, N,N-dimethylacetamide, dimethylformamide, 1,4-dioxane, 2-ethoxyethanol, ethyleneglycol, formamide, hexane, methanol, 2-methoxyethanol, methylbutyl ketone, methylcyclohexane, N-methyl-2-pyrrolidone, nitromethane, pyridine, sulfolane, tetrahydrofuran, tetralin, toluene, 1,1,2-trichloroethene and xylene, and class 3 organic solvents are solvents of the group comprising acetic acid, acetone, anisole, 1-butanol, 2-butanol, butyl acetate, tert-butylmethyl ether, dimethylsulfoxide, ethanol, ethyl acetate, ethyl ether, ethyl formate, formic acid, heptane, isobutyl acetate, isopropyl acetate, methyl acetate, 3-methyl-1-butanol, methylethyl ketone, methylisobutyl ketone, 2-methyl-1-propanol, pentane, 1-pentanol, 1-propanol, 2-propanol and propyl acetate. In particular, rivaroxaban obtained according to the recrystallization or the recrystallization followed by the slurrying of the present invention contains, after drying, less than about 1000 ppm of the solvents selected from dimethylsulfoxide, acetic acid and acetone, and less than about 1000 ppm, preferably less than about 700 ppm, more preferably less than about 400 ppm of acetonitrile.

The residual solvents of rivaroxaban obtained according to the process of the present invention can be quantified by the application of known chromatographic techniques, such as gas chromatography (GC).

Preferably, wet rivaroxaban obtained after recrystallizing and/or slurrying according to the present invention is further dried in a vacuum drier, preferably in a rotary vacuum drier, at a temperature from about 40° C. to about 120° C., preferably from about 50° C. to about 100° C., more preferably from about 60° C. to about 80° C.; and even more preferably at 70±5° C. at a pressure of less than 1013 hPa, preferably less than 700 hPa, more preferably less than 500 hPa, even more preferably less than 250 hPa, and even more preferably less than 100 hPa at any time during the drying process, preferably at the end of the drying process.

Alternatively the wet rivaroxaban obtained after recrystallizing and/or slurrying according to the present invention can be also dried in a tray drier.

In a preferred embodiment of the present invention rivaroxaban is obtained from a 5-chlorothiophene-2-carboxylic acid batch having a reduced amount of some specific impurities, particularly the deschloro impurity thiophene-2-carboxylic acid, the monochloro isomers of 5-chlorothiophene-2-carboxylic acid (i.e. 3-chlorothiophene-2-carboxylic acid and 4-chlorothiophene-2-carboxylic acid) and the dichloro isomers of 5-chlorothiophene-2-carboxylic acid (i.e. 3,4-dichlorothiophene-2-carboxylic acid, 3,5-dichlorothiophene-2-carboxylic acid and 4,5-dichlorothiophene-2-carboxylic acid). Particularly preferred batches of 5-chlorothiophene-2-carboxylic acid are batches of 5-chlorothiophene-2-carboxylic acid having less than about 0.15% (w/w) of any of the deschloro impurity and the monochloro- and the dichloro-isomer impurities, more preferably batches of 5-chlorothiophene-2-carboxylic acid having less than about 0.10% (w/w) of any of the deschloro impurity and the monochloro- and the dichloro-isomer impurities. The authors of the present invention have observed that the presence of impurities, particularly the deschloro, the monochloro and the dichloro derivatives of rivaroxaban, in the final rivaroxaban is directly related to the presence of these impurities in the 5-chlorothiophene-2-carboxylic acid used.

Thus, in a preferred embodiment of the present invention rivaroxaban is obtained by a process comprising the following steps:

(a) (S)-4-{4-[5-(aminomethyl)-2-oxo-1,3-oxazolidin-3-yl]phenyl}morpholin-3-one (compound IV), preferably in form of a salt, reacts with 5-chlorothiophene-2-carbonyl chloride in a solvent, preferably comprising acetone and water, in the presence of an organic base with a pKₐ higher than 5.3, or mixtures thereof, preferably N,N-diisopropylethylamine (DIPEA), wherein the 5-chlorothiophene-2-carbonyl chloride has been previously prepared from a 5-chlorothiophene-2-carboxylic acid having less than about 0.15% (w/w) of any of the deschloro impurity and the monochloro- and the dichloro-isomer impurities, more preferably less than about 0.10% (w/w) of any of the deschloro impurity and the monochloro- and the dichloro-isomer impurities;
(b) recrystallization of the rivaroxaban obtained in step (a) in a mixture comprising dimethylsulfoxide and acetonitrile.

Rivaroxaban according to the present invention can comprise any polymorphic form thereof, any solvate thereof with any solvent, any hydrate thereof or any co-crystal thereof with any suitable coformer.

Preferably rivaroxaban in crystalline form I as described in WO2007039132A2 is obtained by the process according to the present invention. This crystalline form is particularly stable, thus allowing easier further handling like providing the final dosage form without risking additional conversion of the product into impurities or changes of physical characteristics linked to a different polymorphic form like solubility.

In a preferred embodiment of the present invention, the obtained rivaroxaban in crystalline form I as described in WO2007039132A2 is free of other polymorphic or amorphous forms of rivaroxaban. By “free of other polymorphic or amorphous forms” it is meant that 90-100% (w/w), preferably at least 95% (w/w), more preferably at least 99% (w/w) of the product has the desired polymorphic form.

Optically pure rivaroxaban is obtained according to the process of the present invention. The term “optically pure rivaroxaban” refers to rivaroxaban having an optical purity within the range of 99% to 100% (% area by an HPLC method for chiral purity), preferably higher than 99.5% (% area by an HPLC method for chiral purity), more preferably higher than 99.8% (% area by an HPLC method for chiral purity), and even more preferably higher than 99.9% (% area by an HPLC method for chiral purity).

The HPLC method for chiral purity according to the present invention comprises any HPLC method used to measure the optical purity of rivaroxaban, particularly to determine the % area of rivaroxaban and the % area of its enantiomer. Preferably the HPLC method for chiral purity comprises the HPLC method for chiral purity used in the present invention.

The process of the present invention leads to a rivaroxaban of high purity, wherein the rivaroxaban is more than 98.0% (% area) pure when analyzed by an HPLC method for chromatographic purity, preferably more than 99.0% (% area) when analyzed by an HPLC method for chromatographic purity, more preferably more than 99.5% (% area) pure when analyzed by an HPLC method for chromatographic purity, and even more preferably more than 99.8% (% area) pure when analyzed by an HPLC method for chromatographic purity.

The HPLC method for chromatographic purity according to the present invention comprises any HPLC method used to determine the purity of rivaroxaban. Preferably the HPLC method for chromatographic purity comprises the HPLC method for chromatographic purity used in the present invention.

The process of the present invention leads to a rivaroxaban which shows lower amounts of some specific impurities in comparison with the rivaroxaban obtained by the disclosed process in the '823 patent. For example, the process according to the present invention leads to a rivaroxaban free or substantially free of N,N′-bis[{(5S)-2-oxo-3-[4-(3-oxomorpholin-4-yl)phenyl]-1,3-oxazolidin-5-yl}methyl]urea (herein referred as Compound A).

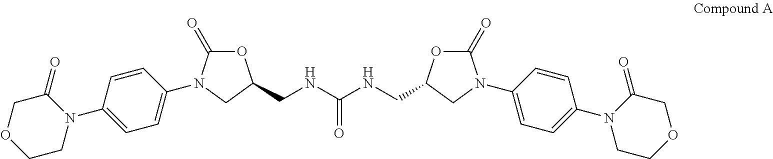 [SMILES: O=C(NC[C@H]1CN(c2ccc(N3CCOCC3=O)cc2)C(=O)O1)NC[C@H]1CN(c2ccc(N3CCOCC3=O)cc2)C(=O)O1]

It should be noted that by “free or substantially free of Compound A” is meant to refer that the rivaroxaban as herein disclosed contains less than the detection limit of Compound A as herein disclosed in an HPLC method for chromatographic purity.

The HPLC method for chromatographic purity according to the present invention comprises any HPLC method used to measure the concentration of Compound A in rivaroxaban. Preferably the HPLC method for chromatographic purity to measure the concentration of Compound A comprises the HPLC method for chromatographic purity used in the present invention.

The term “measure the concentration of Compound A” as used in the present invention comprises quantifying the amount of Compound A with respect to rivaroxaban (w/w) or determining the % area of Compound A.

In a preferred embodiment of the present invention, the detection limit of the Compound A is the detection limit of Compound A in the HPLC method for chromatographic purity used in the present invention, more preferably the detection limit is 0.001% (w/w).

The Compound A according to the present invention can comprise any crystalline or amorphous form thereof, any salt thereof, any solvate thereof with any solvent, any hydrate thereof or any co-crystal thereof with any suitable coformer.

Rivaroxaban obtained according to the process of the present invention is free or substantially free of N,N′-bis[(5S)-2-oxo-3-[4-(3-oxomorpholin-4-yl)phenyl]-1,3-oxazolidin-5-yl}methyl]benzene-1,2-diamide, referred hereinafter as Compound B:

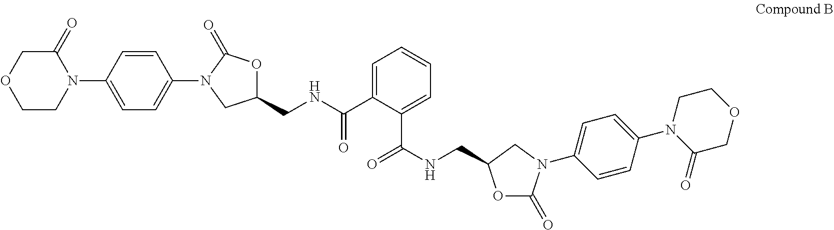 [SMILES: O=C(NC[C@H]1CN(c2ccc(N3CCOCC3=O)cc2)C(=O)O1)c1ccccc1C(=O)NC[C@H]1CN(c2ccc(N3CCOCC3=O)cc2)C(=O)O1]

It should be noted that by “free or substantially free of Compound B” is meant to refer that the rivaroxaban as herein disclosed contains less than 0.15% (w/w) of Compound B as herein disclosed when measured in an HPLC method for chromatographic purity, preferably less than 0.10% (w/w) of Compound B as herein disclosed when measured in an HPLC method for chromatographic purity, more preferably less than 0.05% (w/w) of Compound B as herein disclosed when measured in an HPLC method for chromatographic purity, and even more preferably less than 0.01% (w/w) of Compound B as herein disclosed when measured in an HPLC method for chromatographic purity.

The HPLC method for chromatographic purity according to the present invention comprises any HPLC method used to measure the concentration of Compound B in rivaroxaban. Preferably the HPLC method for chromatographic purity to measure the concentration of Compound B comprises the HPLC method for chromatographic purity used in the present invention.

The term “measure the concentration of Compound B” as used in the present invention comprises quantifying the amount of Compound B with respect to rivaroxaban (w/w) or determining the % area of Compound B.

The Compound B according to the present invention can comprise any crystalline or amorphous form thereof, any salt thereof, any solvate thereof with any solvent, any hydrate thereof or any co-crystal thereof with any suitable coformer.

The present invention also provides a pharmaceutical composition comprising rivaroxaban obtained according to the process of the present invention and one or more pharmaceutically acceptable carrier.

Non-limiting examples of the pharmaceutical composition according to the present invention are oral suspensions, coated tablets, non coated tablets, orodispersible tablets, pellets, pills, granules, capsules, and mini-tablets in capsules.

The term “pharmaceutically acceptable carrier” refers to an excipient, diluent, adjuvant, or carrier with which a compound of the invention is administered.

As used herein, the term “excipient” refers to a pharmaceutically acceptable ingredient that is commonly used in the pharmaceutical technology for preparing granulate and/or solid oral dosage formulations. Examples of categories of excipients include, but are not limited to, binders, disintegrants, lubricants, glidants, stabilizers, fillers and diluents. One of ordinary skill in the art may select one or more of the aforementioned excipients with respect to the particular desired properties of the granulate and/or solid oral dosage form by routine experimentation and without any undue burden. The amount of each excipient used may vary within ranges conventional in the art. The following references which are all hereby incorporated by reference disclose techniques and excipients used to formulate oral dosage forms. See The Handbook of Pharmaceutical Excipients, 4^{th} edition, Rowe et al., Eds., American Pharmaceuticals Association (2003); and Remington: the Science and Practice of Pharmacy, 20^{th} edition, Gennaro, Ed., Lippincott Williams & Wilkins (2000).

In the present description the term “diluent” refers to an excipient which fills out the size of a tablet or capsule, making it practical to produce and convenient for the consumer to use. Suitable diluents include e.g. pharmaceutically acceptable inert fillers, such as microcrystalline cellulose, lactose, dibasic calcium phosphate sugar, sugar alcohols, corn starch, sucrose, silicic anhydride, polysaccharides, N-methylpyrrolidone (Pharmasolve (ISP)) and mixtures thereof. The term sugar and sugar alcohols comprises mannitol, lactose, fructose, sorbitol, xylitol, maltodextrin, dextrates, dextrins, lactitol and mixtures thereof.

As used herein the term “adjuvant” refers to any component which improves the body's response to a pharmaceutical composition.

The term “carrier” refers to a compound that facilitates the incorporation of an active ingredient into the body.

The present invention further relates to a method for the prophylaxis and/or treatment of thromboembolic diseases in a patient comprising administering to said patient a therapeutically effective dose of rivaroxaban according to the present invention.

Non-limiting examples of thromboembolic diseases are cardiac infarct, angina pectoris (including unstable angina), reocclusions and restenoses after an angioplasty or aortocoronary bypass, cerebral infarct, transitory ischemic attacks, peripheral arterial occlusive diseases, pulmonary embolisms or deep venous thromboses.

Another aspect of the present invention relates to the isolated Compound A as herein disclosed.

In further another aspect, the present invention provides a process for preparing N,N′-bis[{(5S)-2-oxo-3-[4-(3-oxomorpholin-4-yl)phenyl]-1,3-oxazolidin-5-yl}methyl]urea (Compound A), said process comprising the reaction of (S)-4-{4-[5-(aminomethyl)-2-oxo-1,3-oxazolidin-3-yl]phenyl}morpholin-3-one (compound IV), or salts thereof, with any carbonyl activated compound selected from the group of phosgene and its synthetic equivalents such as diphosgene or triphosgene, carbonyl diimidazole (CDI) and disuccinimidyl carbonate (DSC), wherein the (S)-4-{4-[5-(aminomethyl)-2-oxo-1,3-oxazolidin-3-yl]phenyl}morpholin-3-one (compound IV) is in excess with respect to the carbonyl activated compound.

In one embodiment of the present invention, the (S)-4-{4-[5-(aminomethyl)-2-oxo-1,3-oxazolidin-3-yl]phenyl}morpholin-3-one (compound IV)/carbonyl activated compound molar ratio is from about 4/1 to about 1.1/1, preferably from about 3/1 to about 1.5/1, more preferably about 2/1.

Preferably the process for preparing Compound A according to the present invention is carried out in the presence of a base in a solvent.

The base used for preparing Compound A according to the present invention comprises any inorganic or any organic base, preferably an organic base selected from the group of pyridine, triethylamine, trimethylamine, tripropylamine, N,N-diisopropylethylamine (DIPEA), N-methylpiperidine, N,N-dimethylaminopyridine (DMAP), N-methylpyrrolidine, 1,4-diazabicyclo[2.2.2]octane (DABCO), 1,5-diazabicyclo[4.3.0]non-5-ene (DBN) and 1,8-diazabicyclo[5.4.0]undec-7-ene (DBU), or mixtures thereof, more preferably triethylamine.

Non-limiting examples of solvents used for preparing the Compound A are ketones such as acetone, methyl ethyl ketone or methyl isobutyl ketone; ethers such as tetrahydrofuran, dioxane, diethylether, diisopropylether, cyclopentyl methyl ether, 2-methyltetrahydrofuran or methyl tert-butyl ether; halogenated solvents such as dichloromethane, chloroform, tetrachloromethane, dichloroethane, chlorobenzene or dichlorobenzene; hydrocarbon aliphatic solvents such as cyclohexane, methylcyclohexane, heptane or hexane; hydrocarbon aromatic solvents such as toluene, benzene, o-xylene, m-xylene or p-xylene; polar aprotic solvents such as N,N-dimethylformamide, acetonitrile, N,N-dimethylacetamide, N-methyl-2-pyrrolidone or dimethylsulfoxide or water or mixtures of two or more of the solvents listed. Preferred solvents used for preparing the Compound A according to the present invention are ethers, more preferably tetrahydrofuran.

Thus, in a preferred embodiment of the present invention Compound A is prepared by reacting (S)-4-{4-[5-(aminomethyl)-2-oxo-1,3-oxazolidin-3-yl]phenyl}morpholin-3-one (compound IV), or salts thereof, with any carbonyl activated compound selected from the group of phosgene and its synthetic equivalents such as diphosgene or triphosgene, carbonyl diimidazole (CDI) and disuccinimidyl carbonate (DSC), wherein the (S)-4-{4-[5-(aminomethyl)-2-oxo-1,3-oxazolidin-3-yl]phenyl}morpholin-3-one (compound IV)/carbonyl activated compound molar ratio is about 2/1 in the presence of an organic base, preferably triethylamine, in a solvent, preferably tetrahydrofuran.

Another aspect of the present invention relates to the isolated Compound B as herein disclosed.

In further another aspect, the present invention provides a process for preparing the Compound B, said process comprising the following steps:

(i) the reaction of (S)-2-({2-oxo-3-[4-(3-oxomorpholin-4-yl)phenyl]-1,3-oxazolidin-5-yl}methyl)-1H-isoindole-1,3(2H)-dione (compound III) with a sulfide salt, an hydrosulfide salt, or hydrogen sulfide, or mixtures thereof to give (S)-2-[({2-oxo-3-[4-(3-oxomorpholin-4-yl)phenyl]-1,3-oxazolidin-5-yl}methyl)carbamoyl]benzoic acid (compound V)

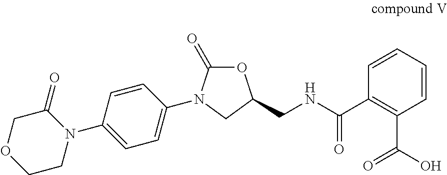 [SMILES: O=C(O)c1ccccc1C(=O)NC[C@H]1CN(c2ccc(N3CCOCC3=O)cc2)C(=O)O1]

(ii) the reaction of the (S)-2-[({2-oxo-3-[4-(3-oxomorpholin-4-yl)phenyl]-1,3-oxazolidin-5-yl}methyl)carbamoyl]benzoic acid (compound V) obtained in step (i) with (s)-4-{4-[5-(aminomethyl)-2-oxo-1,3-oxazolidin-3-yl]phenyl}morpholin-3-one (compound IV), or salts thereof, in the presence of a suitable activating agent.

The sulfide salt used in step (i) for preparing Compound B according to the present invention comprises any inorganic or any organic sulfide salt, preferably an inorganic sulfide salt selected from the group comprising lithium sulfide, sodium sulfide, potassium sulfide, ammonium sulfide, or mixtures thereof, more preferably sodium sulfide.

The hydrosulfide salt used in step (i) for preparing Compound B according to the present invention comprises any inorganic or any organic hydrosulfide salt, preferably an inorganic hydrosulfide salt selected from the group comprising lithium hydrosulfide, sodium hydrosulfide, potassium hydrosulfide, ammonium hydrosulfide, or mixtures thereof, more preferably sodium hydrosulfide.

The activating agent used in step (ii) for preparing Compound B according to the present invention comprises any suitable agent for the activation of carboxylic acids in the formation of amides such as thionyl chloride, N,N′-carbonyldiimidazole (CDI), dicyclohexylcarbodiimide (DCC), diisopropylcarbodiimide (DIC), 1-ethyl-3-(3′-dimethylaminopropyl)carbodiimide (EDC or WSC), 1-propanephosphonic acid cyclic anhydride (T3P), 2-(7-aza-1H-benzotriazole-1-yl)-1,1,3,3-tetramethyluronium hexafluorophosphate (HATU), 2-(1H-benzotriazole-1-yl)-1,1,3,3-tetramethyluronium hexafluorophosphate (HBTU), 2-(6-chloro-1H-benzotriazole-1-yl)-1,1,3,3-tetramethylaminium hexafluorophosphate (HCTU), 2-(1H-benzotriazole-1-yl)-1,1,3,3-tetramethyluronium tetrafluorophosphate (TBTU), benzotriazole-1-yl-oxytris(dimethylamino)phosphonium hexafluorophosphate (BOP), benzotriazole-1-yl-oxytrispyrrolidinophosphonium hexafluorophosphate (PyBOP), (1-cyano-2-ethoxy-2-oxoethylidenaminooxy)dimethylaminomorpholinocarbenium hexafluorophosphate (COMU), or mixtures thereof, more preferably 1-propanephosphonic acid cyclic anhydride (T3P).

Preferably the step (i) and step (ii) in the process for preparing Compound B according to the present invention are carried out in the presence of a solvent, which can be the same for each step (i) and (ii) or, alternatively, the solvent in step (i) can be different from the solvent used in step (ii).

Non-limiting examples of solvents used indistinctly for each step (i) and step (ii) in the process for preparing Compound B according to the present invention are ketones such as acetone, methyl ethyl ketone or methyl isobutyl ketone; ethers such as tetrahydrofuran, dioxane, diethylether, diisopropylether, cyclopentyl methyl ether, 2-methyltetrahydrofuran or methyl tert-butyl ether; esters such as ethyl acetate, methyl acetate, isopropyl acetate, n-propyl acetate, n-butyl acetate, isobutyl acetate, sec-butyl acetate or tert-butyl acetate; halogenated solvents such as dichloromethane, chloroform, tetrachloromethane, dichloroethane, chlorobenzene or dichlorobenzene; hydrocarbon aliphatic solvents such as cyclohexane, methylcyclohexane, heptane or hexane; hydrocarbon aromatic solvents such as toluene, benzene, o-xylene, m-xylene or p-xylene; polar aprotic solvents such as N,N-dimethylformamide, acetonitrile, N,N-dimethylacetamide, N-methyl-2-pyrrolidone or dimethylsulfoxide or water or mixtures of two or more of the solvents listed. Preferred solvents used for preparing the Compound B according to the present invention are mixtures of acetonitrile and water in step (i), and mixtures of acetonitrile and ethyl acetate in step (ii).

Preferably the step (ii) in the process for preparing Compound B according to the present invention is carried out in the presence of a base.

The base used in step (ii) for preparing Compound B according to the present invention comprises any inorganic or any organic base, preferably an organic base selected from the group of pyridine, triethylamine, trimethylamine, tripropylamine, N,N-diisopropylethylamine (DIPEA), N-methylpiperidine, N,N-dimethylaminopyridine (DMAP), N-methylpyrrolidine, 1,4-diazabicyclo[2.2.2]octane (DABCO), 1,5-diazabicyclo[4.3.0]non-5-ene (DBN) and 1,8-diazabicyclo[5.4.0]undec-7-ene (DBU), or mixtures thereof, more preferably triethylamine.

Thus, in a preferred embodiment of the present invention Compound B is prepared by:

(i) reacting (S)-2-({2-oxo-3-[4-(3-oxomorpholin-4-yl)phenyl]-1,3-oxazolidin-5-yl}methyl)-1H-isoindole-1,3(2H)-dione (compound III) with a sulfide salt, preferably sodium sulfide, an hydrosulfide salt, preferably sodium hydrosulfide, or hydrogen sulfide, or mixtures thereof, in a suitable solvent, preferably a mixture of acetonitrile and water, to obtain (S)-2-[({2-oxo-3-[4-(3-oxomorpholin-4-yl)phenyl]-1,3-oxazolidin-5-yl}methyl)carbamoyl]benzoic acid (compound V).
(ii) reacting the (S)-2-[({2-oxo-3-[4-(3-oxomorpholin-4-yl)phenyl]-1,3-oxazolidin-5-yl}methyl)carbamoyl]benzoic acid (compound V) obtained in step (i) with (S)-4-{4-[5-(aminomethyl)-2-oxo-1,3-oxazolidin-3-yl]phenyl}morpholin-3-one (compound IV), or salts thereof, in the presence of a suitable agent for the activation of carboxylic acids in the formation of amides, preferably 1-propanephosphonic acid cyclic anhydride (T3P) and a base, preferably triethylamine, in a suitable solvent, preferably a mixture of acetonitrile and ethyl acetate.

In another aspect, the invention provides a process for determining the suitability for distribution of a batch of rivaroxaban, or a pharmaceutical composition comprising rivaroxaban from said batch, said process comprising:

(a) producing a batch of rivaroxaban, or a pharmaceutical composition comprising rivaroxaban from said batch;
(b) measuring the concentration of Compound A and/or the concentration of Compound B, using respectively Compound A and/or Compound B as reference marker; and
(c) validating the batch for distribution only if the sample of the batch is free or substantially free of Compound A as hereinbefore disclosed and/or free or substantially free of Compound B as hereinbefore disclosed.

In one embodiment of the present invention the process for determining the suitability for distribution of a batch of rivaroxaban, or a pharmaceutical composition comprising rivaroxaban from said batch comprises:

(a) producing a batch of rivaroxaban, or a pharmaceutical composition comprising rivaroxaban from said batch;
(b) measuring the concentration of Compound A using Compound A as reference marker; and
(c) validating the batch for distribution only if the sample of the batch is free or substantially free of Compound A.

In another one embodiment of the present invention the process for determining the suitability for distribution of a batch of rivaroxaban, or a pharmaceutical composition comprising rivaroxaban from said batch comprises:

(a) producing a batch of rivaroxaban, or a pharmaceutical composition comprising rivaroxaban from said batch;
(b) measuring the concentration of Compound B using Compound B as reference marker; and
(c) validating the batch for distribution only if the sample of the batch is free or substantially free of Compound B.

In another embodiment of the present invention the process for determining the suitability for distribution of a batch of rivaroxaban, or a pharmaceutical composition comprising rivaroxaban from said batch, said process comprises:

(a) producing a batch of rivaroxaban, or a pharmaceutical composition comprising rivaroxaban from said batch;
(b) measuring the concentration of Compound A and the concentration of Compound B, using respectively Compound A and Compound B as reference marker; and
(c) validating the batch for distribution only if the sample of the batch is free or substantially free of Compound A and free or substantially free of Compound B.

The production of a batch of rivaroxaban, or a pharmaceutical composition comprising rivaroxaban from said batch of step (a) can be accomplished by any method known in the art.

The measure of the concentration of Compound A and/or Compound B of step (b) can be carried out by means of any suitable analytical method, and preferably is carried out by means of the HPLC method for chromatographic purity used in the present invention or by any equivalent method.

Thus, in a preferred embodiment of the present invention measuring the concentration of Compound A and/or Compound B of step (b) comprises quantifying the amount of Compound A and/or Compound B (w/w) with respect to rivaroxaban (w/w) or determining the % area of Compound A and/or Compound B in the HPLC chromatogram obtained by the HPLC method for chromatographic purity used in the present invention.

Further, there is provided by the present invention an analytical method for determining the purity of a test sample comprising rivaroxaban which comprises:

(a) providing a reference sample comprising (i) rivaroxaban and (ii) one or more reference markers selected from the group consisting of Compound A and Compound B;
(b) carrying out chromatographic separation on said reference sample to obtain a reference chromatographic result relative to said rivaroxaban of each one of the reference marker(s) present in the reference sample;
(c) carrying out chromatographic separation on said test sample to obtain a test chromatographic result; and
(d) comparing the chromatographic results obtained in steps (b) and (c); wherein:
if the test chromatographic result is substantially the same, as the reference chromatographic result Compound A, then the test sample comprises Compound A as an impurity,
if the test chromatographic result is substantially the same, as the reference chromatographic result Compound B, then the test sample comprises Compound B as an impurity.

In one embodiment of the present invention the analytical method for determining the purity of a test sample comprising rivaroxaban comprises:

(a) providing a reference sample comprising (i) rivaroxaban, (ii) Compound A as a reference marker and (iib) Compound B as a reference marker;
(b) carrying out chromatographic separation on said reference sample to obtain a reference chromatographic result relative to said rivaroxaban of each one of the reference marker(s) present in the reference sample;
(c) carrying out chromatographic separation on said test sample to obtain a test chromatographic result; and
(d) comparing the chromatographic results obtained in steps (b) and (c); wherein:
if the test chromatographic result is substantially the same, as the reference chromatographic result Compound A, then the test sample comprises Compound A as an impurity,
if the test chromatographic result is substantially the same, as the reference chromatographic result Compound B, then the test sample comprises Compound B as an impurity.

In another embodiment of the present invention the analytical method for determining the purity of a test sample comprising rivaroxaban comprises:

(a) providing a reference sample comprising (i) rivaroxaban, (ii) Compound A as a reference;
(b) carrying out chromatographic separation on said reference sample to obtain a reference chromatographic result relative to said rivaroxaban of each one of the reference markers present in the reference sample;
(c) carrying out chromatographic separation on said test sample to obtain a test chromatographic result; and
(d) comparing the chromatographic results obtained in steps (b) and (c); wherein:
if the test chromatographic result is substantially the same, as the reference chromatographic result Compound A, then the test sample comprises Compound A as an impurity.

In another embodiment of the present invention the analytical method for determining the purity of a test sample comprising rivaroxaban comprises:

(a) providing a reference sample comprising (i) rivaroxaban, (ii) Compound B as a reference;
(b) carrying out chromatographic separation on said reference sample to obtain a reference chromatographic result relative to said rivaroxaban of each one of the reference markers present in the reference sample;
(c) carrying out chromatographic separation on said test sample to obtain a test chromatographic result; and
(d) comparing the chromatographic results obtained in steps (b) and (c); wherein:
if the test chromatographic result is substantially the same, as the reference chromatographic result Compound B, then the test sample comprises Compound B as an impurity.

The term “reference marker”, as used herein, refers to a compound that is employed in qualitative analysis to confirm the presence of the compound in a sample based on its position in a chromatogram, e.g. in a HPLC or GC chromatogram, or on a Thin Layer Chromatography (TLC) plate. The reference marker compound, optionally in admixture with rivaroxaban, is chromatographed in a first set of chromatographic conditions and its position (reference position) in the chromatogram is noted. Then, the mixture to be analyzed is chromatographed in the same set of chromatographic conditions and the positions of each peak or spot in the chromatogram is recorded (peak/spot positions). When one of the peak/spot positions coincides with the reference position, the mixture is determined to contain at least some reference marker compound.

The term “sample comprising rixaroxaban”, as used herein, refers to a chemical or pharmaceutical mixture containing rivaroxaban in any polymorphic form, or any solvate thereof with any solvent, or any hydrate thereof or any co-crystal thereof with any coformer, intended for pharmaceutical use.

A “reference marker” may also be used for quantitative analysis of rivaroxaban. For example, the HPLC retention time of the reference standard allows a relative retention time with respect to rivaroxaban to be determined, thus making qualitative analysis possible. Furthermore, the concentration of Compound A and/or Compound B in a solution injected into an HPLC or GC column allows the areas under the HPLC or GC peaks to be compared, thus making quantitative analysis possible.

The term “chromatographic result” is used to designate the retention time in GC or HPLC or the relative retention factor in a TLC. Two chromatographic results are considered to be equivalents when the difference between the two results is not more than 10% of the average value of the two results.

Preferably, the chromatographic separation comprises HPLC and as such the above method comprises carrying out the steps of:

(a) providing a reference sample comprising (i) rivaroxaban and (ii) one or more reference markers selected from the group consisting of Compound A and Compound B;
(b) carrying out HPLC on said reference sample to determine the relative retention time compared to the retention time of said rivaroxaban of each one of the reference marker(s) present in the reference sample;
(c) carrying out HPLC on said test sample; and
(d) comparing relative retention times determined in steps (b) and (c); wherein:
if there is observed a relative retention time in step (c) substantially the same as the relative retention time of Compound A, then test sample comprises Compound A as an impurity,
if there is observed a relative retention time in step (c) substantially the same as the relative retention time of Compound B, then test sample comprises Compound B as an impurity.

Alternatively, the chromatographic separation can comprise TLC and as such the above method comprises carrying out the steps of:

(a) providing a reference sample comprising (i) rivaroxaban and (ii) one or more reference markers selected from the group consisting of Compound A and Compound B;
(b) carrying out TLC on said reference sample to determine the relative retention factor on a chromatographic support compared to that of said rivaroxaban of each one of the reference marker(s) present in the reference sample;
(c) carrying out TLC on said test sample; and
(d) comparing the relative retention factors determined in steps (b) and (c); wherein:
if there is observed on said chromatographic support a relative retention factor in step (c) substantially the same as the relative retention factor of Compound A, then test sample comprises Compound A as an impurity,
if there is observed on said chromatographic support a relative retention factor in step (c) substantially the same as the relative retention factor of Compound B, then test sample comprises Compound B as an impurity.

The present invention still further comprises an analytical method for quantifying the purity of a test sample comprising rivaroxaban, which comprises:

(a) providing a test sample of rivaroxaban, containing an unknown concentration of Compound A and/or Compound B;
(b) subjecting said test sample to chromatographic separation;
(c) obtaining a chromatographic quantitative measurement for Compound A and/or Compound B in said test sample; and
(d) calculating the amount of Compound A and/or Compound B in said test sample based on the measurement of step (c) and also on a chromatographic quantitative measurement for Compound A and/or Compound B obtained from at least one reference sample having a known concentration of Compound A and/or Compound B.

In one embodiment of the present invention the analytical method for quantifying the purity of a test sample comprising rivaroxaban comprises:

(a) providing a test sample of rivaroxaban, containing an unknown concentration of Compound A and an unknown concentration of Compound B;
(b) subjecting said test sample to chromatographic separation;
(c) obtaining a chromatographic quantitative measurement for Compound A and Compound B in said test sample; and
(d) calculating the amount of Compound A and Compound B in said test sample based on the measurement of step (c) and also on a chromatographic quantitative measurement for Compound A and Compound B obtained from at least one reference sample having a known concentration of Compound A and Compound B.

In another embodiment of the present invention the analytical method for quantifying the purity of a test sample comprising rivaroxaban comprises:

(a) providing a test sample of rivaroxaban, containing an unknown concentration of Compound A;
(b) subjecting said test sample to chromatographic separation;
(c) obtaining a chromatographic quantitative measurement for Compound A in said test sample; and
(d) calculating the amount of Compound A in said test sample based on the measurement of step (c) and also on a chromatographic quantitative measurement for Compound A obtained from at least one reference sample having a known concentration of Compound A.

In another embodiment of the present invention the analytical method for quantifying the purity of a test sample comprising rivaroxaban comprises:

(a) providing a test sample of rivaroxaban, containing an unknown concentration of Compound B;
(b) subjecting said test sample to chromatographic separation;
(c) obtaining a chromatographic quantitative measurement for Compound B in said test sample; and
(d) calculating the amount of Compound B in said test sample based on the measurement of step (c) and also on a chromatographic quantitative measurement for Compound B obtained from at least one reference sample having a known concentration of Compound B.

In a first embodiment of the above quantification method, the following steps are carried out:

(a) providing a test sample of rivaroxaban, containing an unknown concentration of Compound A and/or Compound B;
(b) providing at least one reference sample having a known concentration of Compound A and/or Compound B;
(c) subjecting said test sample and said reference sample to chromatographic separation;
(d) obtaining chromatographic quantitative measurements for Compound A and/or Compound B in said test sample and said reference sample; and
(e) calculating the amount of Compound A and/or Compound B in said test sample from the measurements of step (d).

More specifically, the following steps are typically carried out in the above first embodiment of the quantification method according to the present invention:

(a) providing a test sample of rivaroxaban, containing an unknown concentration of Compound A and/or Compound B;
(b) providing at least one reference sample having a known concentration of Compound A and/or Compound B;
(c) subjecting said test sample and said reference sample to HPLC;
(d) measuring the area or height of peaks obtained for Compound A and/or Compound B, in said test sample and said reference sample; and
(e) calculating the concentration of Compound A and/or Compound B, in said test sample from the measurements of step (d).

The term “about” when used in the present invention preceding a number and referring to it, is meant to designate any value which lies within the range defined by the number ±10% of its value, preferably a range defined by the number ±5%, more preferably range defined by the number ±2%, still more preferably a range defined by the number ±1%. For example “about 10” should be construed as meaning within the range of 9 to 11, preferably within the range of 9.5 to 10.5, more preferably within the range of 9.8 to 10.2, and still more preferably within the range of 9.9 to 10.1.

All documents referred to herein, including patents, patent applications, and printed publications, are hereby incorporated by reference in their entirety in this disclosure.

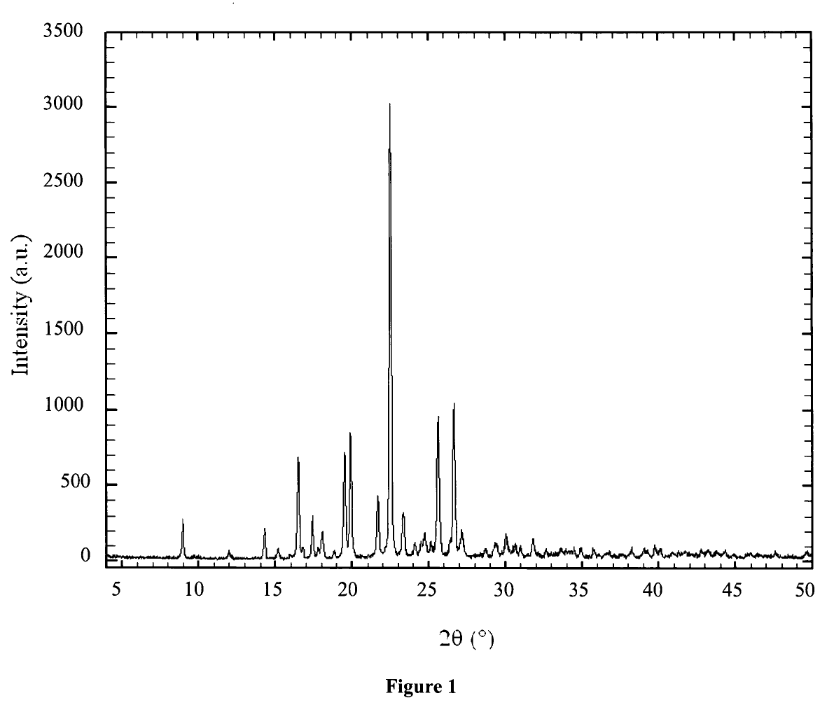

FIG. 1 shows an XRPD plot of rivaroxaban obtained in accordance with Example 2.

### EXAMPLES

The following examples further illustrate the invention but, of course, should not be construed as in any way limiting its scope.

### General Experimental Conditions:

### HPLC Method for Chromatographic Purity:

HPLCs were acquired on a Shimadzu Prominence LC-20 system.

The chromatographic separation was carried out using a Purospher Star RP-18e Endcapped, 5 μm, 4.6 mm×250 mm column, at 28° C.

The mobile phase A was a 0.010M ammonium bicarbonate buffer solution, pH 9.0, which was prepared by dissolving 0.79 g of ammonium bicarbonate in 1000 mL of water, adding 2.0 mL of triethylamine and adjusting pH to 9.0 with formic acid. The mobile phase was mixed, filtered through a 0.22 μm nylon membrane, and degassed.

The mobile phase B was acetonitrile.

The chromatograph was programmed as follows: Initial 0-2 min. 75% mobile phase A, 2-33 min. linear gradient to 34% mobile phase A, 33-36 min. isocratic 34% mobile phase A, 36-48 min. linear gradient to 75% mobile phase A, 48-56 min. isocratic 75% mobile phase A.

The chromatograph was equipped with a 254 nm UV detector. The flow rate was 0.7 mL/min.

Test samples were prepared by dissolving the appropriate amount of sample in 1:1:2 acetonitrile:methanol:mobile phase A (v:v:v), to obtain a concentration of 0.5 mg/mL. 20 μL of the test samples were injected. Chromatograms were run for at least 45 minutes.

Reference standard samples of Compound A and Compound B were prepared by dissolving the appropriate amount of sample in 1:1:2 acetonitrile:methanol:mobile phase A (v:v:v), to obtain a concentration of 0.0005 mg/mL (0.1% with respect to the test sample). 20 μL of the reference standard samples were injected.

### Approximate HPLC Retention Times:

| | | Relative |
|---|---|---|
| | Times | retention |
| Compound | (minutes) | Time |
| Rivaroxaban (compound I) | 21 | 1.00 |
| N,N′-bis[{(5S)-2-oxo-3-[4-(3-oxomorpholin-4- | 6.3 | 0.30 |
| yl)phenyl]-1,3-oxazolidin-5-yl}methyl]urea | | |
| (Compound A) | | |
| N,N′-bis[{(5S)-2-oxo-3-[4-(3-oxomorpholin- | 8.9 | 0.42 |
| 4-yl)phenyl]-1,3-oxazolidin-5-yl}methyl]benzene- | | |
| 1,2-diamide (Compound B) | | |

The limit of detection (LOD) of Compound A: 0.00000504 mg/mL: 0.001% (w/w) with respect to rivaroxaban.
The limit of quantification (LOQ) of Compound A: 0.0000168 mg/mL: 0.0033% (w/w) with respect to rivaroxaban.
The limit of detection (LOD) of Compound B: 0.00000577 mg/mL: 0.0012% (w/w) with respect to rivaroxaban.
The limit of quantification (LOQ) of Compound B: 0.0000192 mg/mL: 0.0038% (w/w) with respect to rivaroxaban.

HPLC Method for Chiral Purity: HPLCs were acquired on a Shimadzu Prominence LC-20 system.

The chromatographic separation was carried out using a Chiralpak IC, 5 μm, 4.6×250 mm column, at 28° C.

The mobile phase was acetonitrile.

The chromatograph was equipped with a 254 nm UV detector. The flow rate was 0.7 mL/min.

Test samples were prepared by dissolving the appropriate amount of sample in acetonitrile to obtain a concentration of 0.5 mg/mL. 20 μL of the test samples were injected. Chromatograms were run for at least 45 minutes.

### GC Method for the Residual Solvents:

### Dimethylsulfoxide for Example 3.

The GC analysis was performed on Shimadzu GC 2010 equipped with a flame ionization detector (FID). The following parameters were used: Carrier gas: He; Column head pressure: 4 psi (constant pressure); Split ratio: 2:0, Injector Temperature: 250° C.; Detector Temperature: 250° C.; Column: TR-WAXDB, Teknokroma 30 m length×0.53 mm internal diameter×1 μm film thickness.

The following temperature program was used: the oven temperature was set at 130° C. for about 20 minutes, then raised to 200° C. with a ramp of 10° C. per minute and maintained at 200° C. for 15 minutes. Injection volume: 2 μL (CombiPal Autosampler).

Standard solutions of dimethylsulfoxide: a stock solution of 433 μg/mL of dimethylsulfoxide in N,N-dimethylformamide was prepared by diluting quantitatively a well known quantity of dimethylsulfoxide. The stock solution of 433 μg/mL of dimethylsulfoxide was quantitatively diluted with N,N-dimethylformamide to obtain standard solutions containing 17 μg/mL and 43 μg/mL of dimethylsulfoxide.

Test solution: 200 mg of rivaroxaban were weighed accurately and dissolved with 10 mL of N,N-dimethylformamide.

### Dimethylsulfoxide for Examples 2, 4 and 5.

The GC analysis was performed on an Agilent 7890A gas chromatograph equipped with a flame ionization detector (FID). The following parameters were used: Carrier gas: He; Column head pressure: 4 psi (constant pressure); Split ratio: 2:1, Injector Temperature: 250° C.; Detector Temperature: 250° C.; Column: TRB-WAX, Teknokroma 30 m length×0.53 mm internal diameter×1 μm film thickness.

The following temperature program was used: equilibration at 130° C. for 5 minutes, the oven temperature was set at 130° C. for about 20 minutes, then raised to 200° C. with a ramp of 10° C. per minute and maintained at 200° C. for 15 minutes. Injection volume: 2 μL (CTC CombiPal Autosampler).

Standard solutions of dimethylsulfoxide: a stock solution of 407 μg/mL of dimethylsulfoxide in N,N-dimethylformamide was prepared by diluting quantitatively a well known quantity of dimethylsulfoxide. The stock solution of 407 μg/mL of dimethylsulfoxide was diluted quantitatively with N,N-dimethylformamide to obtain standard solutions containing 2 μg/mL, 8 μg/mL, 41 μg/mL and 81 μg/mL of dimethylsulfoxide.

Test solution: 100 mg of rivaroxaban were weighed accurately and dissolved with 5 mL of N,N-dimethylformamide.

### Acetonitrile for Examples 2 and 3.

The GC analysis was performed on an Agilent 6890N with a head space Agilent 7694 equipped with a flame ionization detector (FID). The following parameters were used: Carrier gas: He; Column head pressure: 20 psi (constant pressure); Split ratio: 3:0, Injector Temperature: 220° C.; Detector Temperature: 250° C.; Column: VOCOL capillary column, Supelco, 105 m length×0.53 mm internal diameter×3 μm film thickness.

The following temperature program was used: the oven temperature was set at 70° C. for about 16 minutes, then raised to 150° C. with a ramp of 25° C. per minute and maintained at 150° C. for 3 minutes, the raised again to 240° C. with a ramp of 30° C. per minute and maintained at 240° C. for 10 minutes.

Headspace conditions: each sample was heated with shaking for 30 minutes at 100° C. After heating, vials were pressurized with helium at 18 psi for 0.3 min. The sample loop was filled for 0.15 minutes (loop volume: 1 mL) and then injected for 0.5 minutes.

Standard solutions of acetonitrile for example 2: a stock solution of 968 μg/mL of acetonitrile in N,N-dimethylformamide was prepared by diluting quantitatively a well known quantity of acetonitrile. The stock solution of 968 μg/mL of acetonitrile was diluted quantitatively with N,N-dimethylformamide to obtain a solution containing 97 μg/mL of acetonitrile.

Standard solutions of acetonitrile for example 3: a stock solution of 5824 μg/mL of acetonitrile in N,N-dimethylformamide was prepared by diluting quantitatively a well known quantity of acetonitrile. The stock solution of 5824 μg/mL of acetonitrile was diluted quantitatively with N,N-dimethylformamide to obtain a solution containing 5.8 μg/mL, 58 μg/mL and 582 μg/mL of acetonitrile.

Test solution: 100 mg of rivaroxaban were weighed accurately and dissolved with 5 mL of N,N-dimethylformamide.

Procedure: 5.0 mL of the solutions were introduced in vials, suitable for head space injection. The vials were sealed with suitable crimp caps and analyzed.

### Acetonitrile for Examples 4 and 5.

The GC analysis was performed on an Agilent 7890A gas chromatograph equipped with a flame ionization detector (FID) and a Head Space injection auxiliary device. The following parameters were used: Carrier gas: He; Column head pressure: 7.5 psi (constant pressure); Split ratio: 2:1, Injector Temperature: 220° C.; Detector Temperature: 250° C.; Column: VOCOL capillary column, Supelco, 105 m length×0.53 mm internal diameter×3 μm film thickness.

The following temperature program was used: equilibration at 70° C. for 5 minutes, the oven temperature was set at 70° C. for about 16 minutes, then raised to 150° C. with a ramp of 25° C. per minute and maintained at 150° C. for 3 minutes, then raised again to 230° C. with a ramp of 30° C. per minute and maintained at 230° C. for 10 minutes.

Headspace conditions (CTC CombiPal Autosampler): each sample was heated at 100° C. and shaked at 250 rpm for 30 minutes. After heating, the 2.5 ml syringe heated at 120° C. was filled and 1 ml was injected.

Standard solutions of acetonitrile: a stock solution of 189 μg/mL of acetonitrile in N,N-dimethylformamide was prepared by diluting quantitatively a well known quantity of acetonitrile. The stock solution of 189 μg/mL of acetonitrile was diluted quantitatively with N,N-dimethylformamide to obtain standard solutions containing 2 μg/mL, 8 μg/mL and 20 μg/mL of acetonitrile.

Test solution: 100 mg of rivaroxaban were weighed accurately and dissolved with 5 mL of N,N-dimethylformamide.

Procedure: 5.0 mL of the solutions were introduced in 20 ml vials, suitable for head space injection. The vials were sealed with suitable screw caps and analyzed.

### Acetic Acid

The GC analysis was performed on an Agilent 7890A gas chromatograph equipped with a flame ionization detector (FID). The following parameters were used: Carrier gas: He; Column head pressure: 3 psi (constant pressure); Splitless mode, Injector Temperature: 100° C.; Detector Temperature: 300° C.; Column: HP-FFAP capillary column, Agilent, 30 m length×0.53 mm internal diameter×1 μm film thickness.

The following temperature program was used: equilibration at 80° C. for 5 minutes, the oven temperature was set at 80° C. for about 3 minutes, then raised to 150° C. with a ramp of 8° C. per minute and maintained at 150° C. for 5 minutes, then raised again to 230° C. with a ramp of 5° C. per minute and maintained at 230° C. for 10 minutes. Injection volume: 1 μL (CTC CombiPal Autosampler).

Standard solutions of acetic acid: a stock solution of 400 μg/mL of acetic acid in dimethylsulfoxide was prepared by diluting quantitatively a well known quantity of acetic acid. The stock solution of 400 μg/mL of acetic acid was diluted quantitatively with dimethylsulfoxide to obtain standard solutions containing 50 μg/mL and 100 μg/mL of acetic acid.

Test solution: 100 mg of rivaroxaban were weighed accurately and dissolved with 5 mL of dimethylsulfoxide.

### Acetone

The GC analysis was performed on an Agilent 7890A gas chromatograph equipped with a flame ionization detector (FID) and a Head Space injection auxiliary device. The following parameters were used: Carrier gas: He; Column head pressure: 20 psi (constant pressure); Split ratio: 3:1, Injector Temperature: 220° C.; Detector Temperature: 250° C.; Column: VOCOL capillary column, Supelco, 105 m length×0.53 mm internal diameter×3 μm film thickness.

The following temperature program was used: equilibration at 70° C. for 5 minutes, the oven temperature was set at 70° C. for about 16 minutes, then raised to 150° C. with a ramp of 25° C. per minute and maintained at 150° C. for 3 minutes, the raised again to 230° C. with a ramp of 30° C. per minute and maintained at 230° C. for 10 minutes.

Headspace conditions (CTC CombiPal Autosampler): each sample was heated at 100° C. and shaked at 250 rpm for 30 minutes. After heating, the 2.5 ml syringe heated at 120° C. was filled and 1 ml was injected.

Standard solutions of acetone: a stock solution of 100 μg/mL of acetone in N,N-dimethylformamide was prepared by diluting quantitatively a well known quantity of acetone. The stock solution of 100 μg/mL of acetone was diluted quantitatively with N,N-dimethylformamide to obtain standard solutions containing 1 μg/mL and 2 μg/mL of acetone.

Test solution: 100 mg of rivaroxaban were weighed accurately and dissolved with 5 mL of N,N-dimethylformamide.

Procedure: 5.0 mL of the solutions were introduced in 20 ml vials, suitable for head space injection. The vials were sealed with suitable screw caps and analyzed.

### X-Ray Powder Diffraction (XRPD):

XRPD patterns were recorded on a Siemens D5000 diffractometer equipped with two symmetrically mounted vertical goniometers (Bragg-Brentano geometry) with horizontal sample stages, a X-ray tube, a high voltage generator (working at 45 kV and 35 mA) and standard scintillation detectors. Ni-filtered Cu-anode source was used and diffracted radiation was further monochromatized with a graphite crystal to avoid fluorescence effects ((λ(K_{α})=1.54056 Å). Routine diffraction patterns were recorded including values of 2θ that range from 4 to 50° with a sampling rate of 0.02° per second and a step time of 1 second per step. Powdered samples were pressed between two glass plates, forming a film. DIFFRAC Plus measurement software with EVA evaluation software (Bruker) was used to record the data and for a primary analysis of the diffraction patterns. The equipment was periodically calibrated using quartz and silicon.

### REFERENCE EXAMPLES

### Preparation of Reactants

### Reference Example 1

### Synthesis of (S)-4-{4-[5-(aminomethyl)-2-oxo-1,3-oxazolidin-3-yl]phenyl}morpholin-3-one sulfate (2:1) [compound (2:1)]

26.07 g (61.9 mmol) of (S)-2-({2-oxo-3-[4-(3-oxomorpholin-4-yl)phenyl]-1,3-oxazolidin-5-yl}methyl)-1H-isoindole-1,3(2H)-dione (compound III, obtained from compound II following the process disclosed in the '823 patent) were suspended in 196 mL of ethanol. 24.0 mL (278.4 mmol) of 40% w/w aqueous methylamine were added to the suspension, and the resulting mixture was heated to 60-65° C. and maintained at this temperature for about 2.5 hours. The content of unreacted compound III was checked to be below 5% by TLC. At this point, 75.1 g (178.2 mmol) of 20% w/w aqueous sulfuric acid were added over the reaction mixture while keeping the temperature above 60° C. Precipitation was observed during the addition. The resulting suspension was heated to 70-75° C. and stirred at this temperature for about 1 hour, then cooled down to 20-25° C. and stirred at this temperature for about 1 hour. Subsequently, the suspension was filtered and the collected solid was washed with 26.1 mL of ethanol followed by 26.1 mL of acetone to give 18.4 g of wet (S)-4-{4-[5-(aminomethyl)-2-oxo-1,3-oxazolidin-3-yl]phenyl}morpholin-3-one sulfate (2:1) as a white solid. Estimated dry mass: 17.4 g. Yield: 82.7%.

### Reference Example 2

### Synthesis of 5-chlorothiophene-2-carbonyl chloride

10.6 g (65.2 mmol) of 5-chlorothiophene-2-carboxylic acid were suspended in 31.8 mL of toluene. The suspension was heated to 75-80° C., and 5.7 mL (78.2 mmol) of thionyl chloride were added dropwise to the stirred suspension while keeping the temperature at 75-80° C. The addition vessel was rinsed with 3.2 mL of toluene. The resulting clear, deep orange solution was stirred for about 30 minutes at 75-80° C., and then heated to reflux for about 30 minutes. At this point, 42.4 mL of toluene were added to the stirred solution, and the resulting mixture was concentrated by distilling off 45.6 mL of toluene under vacuum without exceeding 65° C., to give an orange solution of 5-chlorothiophene-2-carbonyl chloride in toluene, which was used directly in the next step of the synthesis.

### Reference Example 3

### Synthesis of (S)-4-{4-[5-(aminomethyl)-2-oxo-1,3-oxazolidin-3-yl]phenyl}morpholin-3-one hydrochloride (compound IV.hydrochloride)

26.00 g (79.3 mmol) of (S)-2-({2-oxo-3-[4-(3-oxomorpholin-4-yl)phenyl]-1,3-oxazolidin-5-yl}methyl)-1H-isoindole-1,3(2H)-dione (compound III, obtained from compound II following the process disclosed in the '823 patent) were suspended in 95.0 mL of ethanol. 21.1 mL (271.6 mmol) of 40% w/w aqueous methylamine were added to the suspension, and the resulting mixture was heated to 60-63° C. and maintained at this temperature for about 2 hours. The content of unreacted compound III was checked to be below 5% by TLC. After cooling to 55-60° C., a total of 31.45 g (172.3 mmol) of 20% w/w aqueous hydrochloric acid were added over the reaction mixture until the pH was 2.65. Precipitation was observed during the addition. The resulting suspension was cooled down to 20° C. and subsequently filtered. The collected solid was washed with 15.0 mL of methanol to give 20.01 g of wet (S)-4-{4-[5-(aminomethyl)-2-oxo-1,3-oxazolidin-3-yl]phenyl}morpholin-3-one hydrochloride as a white solid. After drying at 60° C., 17.49 g of dry product were obtained. Yield: 86.5%.

### Reference Example 4

### Synthesis of 5-chlorothiophene-2-carbonyl chloride

10.01 g (61.5 mmol) of 5-chlorothiophene-2-carboxylic acid were suspended in 32.7 mL of toluene. The suspension was heated to 75-80° C., and 5.4 mL (78.2 mmol) of thionyl chloride were added dropwise to the stirred suspension while keeping the temperature at 75-80° C. The resulting clear, deep orange solution was stirred for 30 minutes at 75-80° C., and then heated to reflux for about 30 minutes. At this point the solution was cooled down to less than 60° C. and the mixture was concentrated by distilling off 2.7 mL of toluene under vacuum without exceeding 60° C. This gave an orange solution of 5-chlorothiophene-2-carbonyl chloride in toluene, which was used directly in the next step of the synthesis.

### Examples of the Invention

### Example 1

### Synthesis of Rivaroxaban (Compound I)

16.4 g (dry mass, 24.1 mmol) of (S)-4-{4-[5-(aminomethyl)-2-oxo-1,3-oxazolidin-3-yl]phenyl}morpholin-3-one sulfate (2:1) [compound IV.sulfate (2:1)], as obtained in reference example 1, were suspended in a mixture of 83.8 mL of water and 29.6 mL of acetone. After cooling down to 5-10° C., 20.2 mL (115.9 mmol) of N,N-diisopropylethylamine were added dropwise while keeping the temperature within the range of 5-10° C. The addition vessel was rinsed with 8.2 mL of acetone. The solution of 5-chlorothiophene-2-carbonyl chloride (65.2 mmol) in toluene obtained in reference example 2 was added at this point over the resulting solution, while maintaining the temperature in the range of 5-10° C. Copious precipitation was observed during the addition. The addition vessel was washed down with 7.4 mL of toluene. The resulting creamy, off-white suspension was heated to 20-25° C. and stirred at this temperature for about 1 hour. Then, 37.8 mL of acetone were added and the resulting suspension was heated to 45-50° C. and stirred at this temperature for about 30 minutes. After cooling down to 20-25° C. and stirring for about 1 hour, the suspension was filtered and washed twice with 16.4 mL of acetone to give 21.0 g of wet, crude rivaroxaban as an off-white solid. Estimated dry mass: 20.6 g. Yield: 97.9%.

HPLC chromatographic purity: Rivaroxaban: 98.866% (% area); Compound A: not detected; Compound B: 0.119% (% area). HPLC chiral purity: 99.998% (% area).

### Example 2

### Recrystallization of Rivaroxaban (Compound I)

20.6 g (estimated dry mass) of crude rivaroxaban as obtained in Example 1 were suspended in a mixture of 103 mL of acetonitrile and 61.8 mL of dimethylsulfoxide. The suspension was heated to reflux (about 95° C.) until complete dissolution was observed. The solution was then cooled down to 5-10° C. and stirred at this temperature for about 1 hour. The resulting suspension was filtered and washed with 20.6 mL of acetonitrile to give 18.1 g of wet rivaroxaban.

HPLC chromatographic purity: Rivaroxaban: 99.658% (% area); Compound A: not detected; Compound B: 0.045% (% area).

The solid was suspended in a mixture of 87.0 mL of acetonitrile and 52.2 mL of dimethylsulfoxide. The suspension was heated to reflux (about 95° C.) until complete dissolution was observed. The hot solution was filtered to remove insoluble particles, and the filter was washed with 1.7 mL of acetonitrile. The solution was then cooled down to 5-10° C. and stirred at this temperature for about 1 hour. The resulting suspension was filtered and washed with 17.4 mL of acetonitrile. The wet solid was dried at 60° C. under vacuum to give 15.9 g of rivaroxaban as a white solid. Yield: 77.2%.

HPLC chromatographic purity: Rivaroxaban: 99.883% (% area); Compound A: not detected; Compound B: 0.010% (w/w). HPLC chiral purity: 99.999% (% area). Residual DMSO (GC): 806 ppm. Residual acetonitrile (GC): 357 ppm. XRPD: Form I (see FIG. 1).

### Example 3

### Recrystallization of Rivaroxaban (Compound I)

1.500 Kg of wet, crude rivaroxaban (obtained as disclosed in Example 1, estimated dry mass 1.295 Kg) were mixed with 5.0 Kg of dimethylsulfoxide and 0.1 Kg of decolorizing charcoal. The suspension was heated to about 95° C. until complete dissolution occurred. The hot solution was stirred at this temperature and was filtered to remove charcoal and insoluble particles. The filter was washed with 1.0 Kg of dimethylsulfoxide.

6.0 Kg of acetonitrile were added to the filtered solution. The resulting mixture was refluxed until complete dissolution and then cooled down to 5-10° C. over 30 minutes. The resulting suspension was then stirred at this temperature for 150 minutes. The suspension was filtered and the collected off-white solid was washed with 2.5 Kg of acetonitrile, to give 1.273 Kg of wet, white solid.

The wet solid was dried under vacuum for 6 hours at about 65-75° C. to give 0.930 Kg of dry rivaroxaban (yield: 71.8%).

The dry rivaroxaban was then subjected to a micronizing process by jet milling.

HPLC chromatographic purity: Rivaroxaban: 99.919% (% area); Compound A: not detected; Compound B: 0.023% (w/w). HPLC chiral purity 100% (% area). Residual DMSO (GC): 862 ppm. Residual acetonitrile (GC): 368 ppm. XRPD: Form I (substantially equivalent to FIG. 1).

### Example 4

### Recrystallization of Rivaroxaban (Compound I)

8.00 g (18.4 mmol) of the dry rivaroxaban, as obtained in Example 3 were suspended in a mixture of 24 mL of dimethylsulfoxide and 40 mL of acetonitrile. The resulting mixture was heated to 90-95° C. and maintained at this temperature until complete dissolution. The solution was cooled slowly to 0-5° C. at a rate of approximately 15° C. per hour. The resultant suspension was stirred at this temperature for about 1 hour. Subsequently, the suspension was filtered and the collected solid was washed with 8 mL of acetonitrile to give 7.40 g of wet rivaroxaban as a white solid. The solid was dried at 70° C. under vacuum for 5 hours. Dry mass: 7.32 g. Yield: 91.5%.

HPLC chromatographic purity: Rivaroxaban: 99.945% (% area); Compound A: not detected; Compound B: 0.006% (w/w). HPLC chiral purity: 100% (% area). Residual acetonitrile (GC): 116 ppm. Residual DMSO: 332 ppm. XRPD: Form I (substantially equivalent to FIG. 1).

### Example 5

### Recrystallization of Rivaroxaban (Compound I)

8.00 g (18.4 mmol) of the dry rivaroxaban, as obtained in Example 3, were suspended in a mixture of 24 mL of dimethylsulfoxide and 40 mL of acetonitrile. The resulting mixture was heated to 90-95° C. and maintained at this temperature until complete dissolution. The solution was cooled to 88° C. and 10 mg of rivaroxaban were added to seed the solution. The mixture was cooled to 85° C. and held at this temperature for 3.5 hours. Subsequently, it was cooled to 70° C. over 3 hours. The suspension was then cooled to 0-5° C. over 1.5 hours. The resultant suspension was stirred at this temperature for about 1 hour. Subsequently, the suspension was filtered and the collected solid was washed with 8 mL of acetonitrile to give 7.33 g of wet rivaroxaban as a white solid. The solid was dried at 70° C. under vacuum for 5 hours. Dry mass: 7.23 g. Yield: 91.4%.

HPLC chromatographic purity: Rivaroxaban 99.949% (% area); Compound A; not detected; Compound B: 0.005% (w/w). HPLC chiral purity: 100% (% area). Residual acetonitrile (GC): 48 ppm; Residual DMSO: 307 ppm. XRPD: Form I (substantially equivalent to FIG. 1).

### Example 6

### Recrystallization of Rivaroxaban (Compound I)

8.00 g (18.4 mmol) of the dry rivaroxaban, as obtained in Example 3, were suspended in 48 mL of glacial acetic acid. The resulting mixture was heated to 105-110° C. and maintained at this temperature until complete dissolution. The solution was cooled to 20-25° C. over 1 hour. The resultant suspension was stirred at this temperature for about 10 minutes. Subsequently, the suspension was filtered and the collected solid was washed twice with 16 mL of acetone to give 7.54 g of wet rivaroxaban as a white solid. The solid was dried at 70° C. under vacuum for 5 hours. Dry mass: 7.53 g. Yield: 94.1%.

HPLC chromatographic purity: Rivaroxaban: 99.930% (% area), Compound A: not detected; Compound B: 0.012% (w/w). HPLC chiral purity: 100% (% area). Residual acetic acid (GC): 363 ppm. Residual acetone (GC): 2 ppm. XRPD: Form I (substantially equivalent to FIG. 1).

### Example 7

### Recrystallization of Rivaroxaban (Compound I)

8.00 g (18.4 mmol) of the dry rivaroxaban, as obtained in Example 3, were suspended in 48 mL of glacial acetic acid. The resulting mixture was heated to 105-110° C. and maintained at this temperature until complete dissolution. The solution was cooled to 20-25° C. over 1 hour. The resultant suspension was stirred at this temperature for about 10 minutes, filtered and the collected solid was re-suspended in 40 mL of acetone. The resulting suspension was stirred at 20-25° C. for about 1 hour. Subsequently, the suspension was filtered and washed twice with 8 mL of acetone to give 6.86 g of wet rivaroxaban as a white solid. The solid was dried at 70° C. under vacuum for 5 hours. Dry mass: 6.86 g. Yield: 85.8%.

HPLC chromatographic purity: Rivaroxaban: 99.941% (% area); Compound A: not detected; Compound B: 0.011% (w/w). HPLC chiral purity: 100% (% area). Residual acetic acid (GC): 330 ppm. Residual acetone (GC): 23 ppm. XRPD: Form I (substantially equivalent to FIG. 1).

### Example 8

### Preparation of N,N′-bis[{(5S)-2-oxo-3-[4-(3-oxomorpholin-4-yl)phenyl]-1,3-oxazolidin-5-yl}methyl]urea (Compound A)

1.23 g (12.3 mmol) of triethylamine, 4.0 g (12.3 mmol) of (S)-4-{4-[5-(aminomethyl)-2-oxo-1,3-oxazolidin-3-yl]phenyl}morpholin-3-one (compound IV) and 500 mL of tetrahydrofuran were mixed together in a 1000 mL reactor and cooled down in an ice bath for 10 minutes. 0.60 g (6.1 mmol) of triphosgene were added and the resulting mixture was stirred for 2 hours. Another 1.23 g (12.3 mmol) of triethylamine were added and the mixture was stirred overnight at room temperature. 200 mL of saturated sodium carbonate solution were added and two phases separated. The organic phase was collected and stirred with 200 mL of brine. The mixture was filtered and the collected solid was washed with 30 mL of ethyl acetate to give 1.44 g of white solid. Yield: 38.8%. HPLC chromatographic purity: 96.98% (% area).

Analytical data: m.p.: 241.0-241.8° C.; IR (KBr, cm⁻¹): 3520, 3374, 3308, 3144, 3111, 3066, 3049, 2972, 2943, 2872, 1749, 1726, 1664, 1649, 1607, 1522, 1477, 1435, 1414, 1385, 1346, 1329, 1285, 1265, 1233, 1163, 1146, 1121, 1098, 1061, 1028, 993, 964, 924, 835, 779, 754, 725, 711, 691, 644, 613; ¹H NMR (400 MHz, DMSO-d₆, ppm): 7.55 (dm, J=9.0, 4H), 7.39 (dm, J=9.0, 4H), 6.51 (t, J=5.9, 2H), 4.62 (m, 2H), 4.17 (s, 4H), 4.07 (t, J=8.9, 2H), 3.95 (app. t, J=5.2, 4H), 3.77 (dd, J=6.2, J=9.0, 2H), 3.70 (app. t, J=5.2, 4H), 3.37 (t, J=5.4, 4H). ¹³C NMR (101 MHz, DMSO-d₆, ppm): 165.9, 158.4, 154.2, 137.0, 136.5, 125.9, 118.3, 72.1, 67.7, 63.5, 49.0, 47.1, 42.2; MS (direct infusion, ESI+), m/z (Da) and relative abundance (%):626 ([M+NH₄]⁺, 6), 609 ([M+H]⁺, 2), 179 (81), 174 (63), 157 (100).

### Example 9

### Preparation of N,N′-bis[{(5S)-2-oxo-3-[4-(3-oxomorpholin-4-yl)phenyl]-1,3-oxazolidin-5-yl}methyl]benzene-1,2-diamide (Compound B)

20.0 g (23.80 mmol) of (S)-2-({2-oxo-3-[4-(3-oxomorpholin-4-yl)phenyl]-1,3-oxazolidin-5-yl}methyl)-1H-isoindole-1,3(2H)-dione (compound III, obtained from compound II following the process disclosed in the '823 patent) were suspended in 600 mL of acetonitrile and cooled to 0° C. A solution of 3.6 g (7.20 mmol) of sodium sulfide in 60 mL of water was added dropwise to the suspension over 1 hour. The reaction mixture was then stirred for 30 minutes between 5-10° C. The pH was adjusted to 7 using 1.5 M hydrochloric acid solution whilst maintaining the temperature at 5-10° C. The mixture was filtered and 40.0 g of sodium chloride were added to the filtrate. The aqueous phase was collected and kept aside. The organic phase and the solid obtained from the filtration were loaded into the reactor and cooled to 0° C. A solution of 3.6 g (7.20 mmol) of sodium sulfide in 60 mL of water was added dropwise to the suspension over 1 hour. The reaction mixture was then stirred for 30 minutes between 5-10° C. The pH was adjusted to 7 using 1.5 M hydrochloric acid solution whilst maintaining the temperature at 5-10° C. The mixture was filtered and 40.0 g of sodium chloride were added to the filtrate. The aqueous phase was collected and combined with the previous one which was kept aside. The combined aqueous phase was cooled to 0° C. The pH was adjusted to 4-5 using 1.5 M hydrochloric acid solution whilst maintaining the temperature at 5-10° C. 40.0 g of sodium chloride were added and the solution was extracted twice with 600 mL of acetonitrile. The combined organic phases were dried with 60 g of anhydrous sodium sulfate, filtered and concentrated under vacuum without exceeded 38° C. 11.0 g of (S)-2-[({2-oxo-3-[4-(3-oxomorpholin-4-yl)phenyl]-1,3-oxazolidin-5-yl}methyl)carbamoyl]benzoic acid (compound V) were obtained as a pale yellow foam-like solid. Yield: 56.0%. HPLC chromatographic purity: 95.29% (% area).

10.0 g (22.8 mmol) of (S)-2-[({2-oxo-3-[4-(3-oxomorpholin-4-yl)phenyl]-1,3-oxazolidin-5-yl}methyl)carbamoyl]benzoic acid (compound V) and 7.3 g (25.0 mmol) of (S)-4-{4-[5-(aminomethyl)-2-oxo-1,3-oxazolidin-3-yl]phenyl}morpholin-3-one hydrochloride (compound IV), obtained as disclosed in the '823 patent, were suspended in 1100 mL of acetonitrile. The mixture was cooled to 5° C. and 9.2 g (91.0 mmol) of triethylamine were added dropwise to the suspension. The suspended solid dissolved (pH about 8) and was stirred for 10 minutes after the addition was complete. Then 17.4 g (27.3 mmol) of 1-propanephosphonic acid cyclic anhydride (T3P, 50 wt. % dissolved in ethyl acetate) was slowly added dropwise to the solution and the resultant mixture was stirred for 2 hours. The product precipitated. It was filtered and the solid was washed twice with 30 mL of acetonitrile and dried. 10.2 g of N,N-bis[{(5S)-2-oxo-3-[4-(3-oxomorpholin-4-yl)phenyl]-1,3-oxazolidin-5-yl}methyl]benzene-1,2-diamide (Compound B) were obtained as a grey solid. Yield: 62.8%. HPLC chromatographic purity: 95.47% (% area).

A 9.52 g sample of the above obtained Compound B was suspended in 47.6 mL of acetonitrile (5 volumes) and 38.1 mL of dimethylsulfoxide (4 volumes). The suspension was heated to reflux and was then cooled to 0-10° C. The mixture was stirred at this temperature for 2 hours and then filtered. The collected solid was washed with two portions of 9.5 mL of acetonitrile and was then dried in a vacuum oven. 8.22 g of N-bis[{(5S)-2-oxo-3-[4-(3-oxomorpholin-4-yl)phenyl]-1,3-oxazolidin-5-yl}methyl]benzene-1,2-diamide (Compound B) was obtained as an off-white solid. Yield: 86.3%. HPLC chromatographic purity: 97.71% (% area).

Analytical data: m.p.: 203.8-204.2° C.; IR (KBr, cm⁻¹): 3250, 3055, 2982, 2953, 2928, 2872, 1744, 1724, 1663, 1640; 1522, 1474, 1431, 1414, 1344, 1316, 1288, 1231, 1163, 1148, 1121, 1109, 1069, 1028, 1015, 995, 986, 953, 922, 891, 864, 833, 799, 775, 756, 725, 712, 696, 671, 615; ¹H NMR (400 MHz, DMSO-d₆, ppm): 8.73 (t, J=5.8, 2H), 7.58 (dm, J=9.2, 4H), 7.43-7.49 (m, 4H), 7.39 (dm, J=9.2, 4H), 4.82 (m, 2H), 4.11 (t, J=9.0, 2H), 4.19 (s, 4H), 3.97 (t, 2H), 3.97 (app.t, J=5.0, 2H), 3.70 (app.t, J=5.0, 4H), 3.50-3.63 (m, 4H); ¹³C NMR (101 MHz, DMSO-d₆, ppm): 169.0 (C), 165.9 (C), 154.2 (C), 136.9 (C), 136.6 (C), 136.0 (C), 129.5 (CH), 127.4 (CH), 125.8 (CH), 118.3 (CH), 71.4 (CH), 67.7 (CH₂), 63.5 (CH₂), 48.9 (CH₂), 47.3 (CH₂), 41.4 (CH₂); Elemental analysis: C₃₆H₃₆N₆O₁₀ (712.71 g/mol): calculated C, 60.67%; H, 5.09%; N, 11.79%. found: C, 60.65%; H, 5.01%; N, 11.84%. MS (direct infusion, ESI+): m/z (Da) and relative abundance (%): 714 (44), 713 ([M+H]⁺, 100), 680 (69), 475 (47), 453 (73).

### Comparative Examples

### Comparative Example 1

### Synthesis of Rivaroxaban (Compound I) as is Disclosed in '823 Patent

6.80 g sodium carbonate were mixed with 87.0 mL of water and the suspension was stirred at room temperature until all solid dissolved. The solution was then cooled down to 10° C. 17.00 g (51.9 mmol) of (S)-4-{4-[5-(aminomethyl)-2-oxo-1,3-oxazolidin-3-yl]phenyl}morpholin-3-one hydrochloride as obtained in reference example 3, 5.1 mL water and 39.5 mL acetone were then successively added to the solution to give a solution. While keeping the mixture at 8-12° C., 37.15 g of 5-chlorothiophene-2-carbonyl chloride (−30% strength in toluene), as obtained in reference example 4, were added. Following this, 7.7 mL of toluene were added. The mixture so formed was then heated to 50° C. A further 39.6 mL of acetone were added and the mixture was stirred for another 30 minutes at 50-53° C. After cooling down to 26° C. the mixture was filtered and washed with 15.0 mL water and 15.0 mL acetone to give 23.62 g of wet, crude rivaroxaban as an off-white solid. Estimated dry mass: 21.06 g. Yield: 93.12%.

HPLC chromatographic purity: Rivaroxaban: 98.378% (% area). Compound A: 0.023% (w/w); Compound B: 0.108% (% area). HPLC chiral purity: 99.998% (% area).

### Comparative Example 2

### Recrystallization of Rivaroxaban (Compound I) in Acetic Acid as is Disclosed in '823 Patent

20.33 g (estimated dry mass) of crude rivaroxaban were suspended in 121.0 mL glacial acetic acid. The suspension was heated to reflux (about 102° C.) until complete dissolution was observed. The solution was stirred at this temperature for 10 minutes and then, following a hot filtration to remove insoluble particles, cooled down to 20° C. The resulting suspension was filtered and washed with 20.0 mL of acetic acid and 20.0 mL water to give 25.1 g of wet rivaroxaban. The wet solid was dried at 60° C. under vacuum for 4 hours to give 16.17 g of rivaroxaban as a white solid. Yield: 79.6%.

HPLC chromatographic purity: Rivaroxaban: 99.694% (% area). Compound A: 0.002% (% area), Compound B: 0.027% (w/w), HPLC chiral purity: 100% (% area), XRFD: Form I (substantially equivalent to FIG. 1).

### Comparative Example 3

### Synthesis of Rivaroxaban (Compound I)

28.1 mL of water were added to 5.50 g (dry mass, 8.08 mmol) of (S)-4-{4-[5-(aminomethyl)-2-oxo-1,3-oxazolidin-3-yl]phenyl}morpholin-3-one sulfate (2:1) [compound IV.sulfate (2:1)], as obtained in reference example 1, resulting in a white suspension. 3.92 g of triethylamine (38.78 mmol) were added dropwise at room temperature, followed by 12.8 mL of acetone. The resultant solution was then cooled to 5-10° C. A solution of 5-chlorothiophene-2-carbonyl chloride (21.83 mmol) in toluene, as obtained in reference example 2, was added dropwise while maintaining the temperature in the range of 5-10° C. Precipitation was observed during the addition. Once the addition was complete, 2.5 mL of toluene were added to the suspension. The suspension was then heated to room temperature (20° C.) and stirred at this temperature for about 1 hour. This was then heated to 45-50° C. upon which 12.7 mL of acetone was added. The suspension was heated to 45-50° C. and stirred at this temperature for about 30 minutes. After cooling down to 20-25° C. and stirring at this temperature for about 1 hour, the suspension was filtered to give 6.36 g of wet crude rivaroxaban as a white solid. Estimated dry mass: 5.66 g. Yield: 80.3%.

5.74 g of wet, crude rivaroxaban from the previous step were mixed with 25.5 mL of acetonitrile and 20.4 mL of dimethylsulfoxide and heated to reflux. Once completely dissolved, the solution was cooled down to 20-25° C. and stirred at this temperature for 1 hour. Filtration of the suspension gave 4.32 g of wet rivaroxaban as a white solid.

HPLC chromatographic purity: 99.408% (% area).

3.95 g of wet, crude rivaroxaban from the previous recrystallization step were mixed with 15.3 mL of glacial acetic acid and heated to reflux. Once completely dissolved, the solution was cooled down to 20-25° C. over and stirred at this temperature for 30 minutes. Filtration of the suspension gave 3.63 g of wet rivaroxaban as a white solid.

HPLC chromatographic purity: 99.715% (% area).

1.-45. (canceled)

46. A process for determining the suitability for distribution of a batch of rivaroxaban (compound I), or a pharmaceutical composition comprising rivaroxaban, said process comprising:
(a) producing a batch of rivaroxaban, or a pharmaceutical composition comprising rivaroxaban;
(b) measuring in a sample of the batch or the pharmaceutical composition the concentration of N,N′-bis[{(5S)-2-oxo-3-[4-(3-oxomorpholin-4-yl)phenyl]-1,3-oxazolidin-5-yl}methyl]urea (Compound A)
and/or the concentration of N,N′-bis[(5S)-2-oxo-3-[4-(3-oxomorpholin-4-yl)phenyl]-1,3-oxazolidin-5-yl}methyl]benzene-1,2-diamide (Compound B) using, respectively, Compound A and/or Compound B as reference marker; and
(c) validating the batch or the pharmaceutical composition for distribution only if the sample of the batch or the pharmaceutical composition is free or substantially free of Compound A and/or free or substantially free of Compound B.

47. The process according to claim 46, wherein the batch or the pharmaceutical composition is only validated for distribution when it contains less than 0.001% (w/w) of Compound A.

48. The process according to claim 46, wherein the batch or the pharmaceutical composition is only validated for distribution when it contains less than 0.15% (w/w) of Compound B.

49. The process according to claim 48, wherein the batch or the pharmaceutical composition is only validated for distribution when it contains less than 0.10% (w/w) of Compound B.

50. The process according to claim 49, wherein the batch or the pharmaceutical composition is only validated for distribution when it contains less than 0.05% (w/w) of Compound B.

51. The process according to claim 50, wherein the batch or the pharmaceutical composition is only validated for distribution when it contains less than 0.01% (w/w) of Compound B.

52. A process for preparing a batch of rivaroxaban (compound I) for pharmaceutical use, or a pharmaceutical composition comprising rivaroxaban, said process comprising determining the suitability for distribution of the batch of rivaroxaban, or the pharmaceutical composition comprising rivaroxaban, by a process comprising:
(a) producing a batch of rivaroxaban, or a pharmaceutical composition comprising rivaroxaban;
(b) measuring in a sample of the batch or the pharmaceutical composition the concentration of N,N′-bis[{(5S)-2-oxo-3-[4-(3-oxomorpholin-4-yl)phenyl]-1,3-oxazolidin-5-yl}methyl]urea (Compound A) using Compound A as reference marker; and
(c) validating the batch or the pharmaceutical composition for distribution only if the sample of the batch or the pharmaceutical composition is free or substantially free of Compound A.

53. The process according to claim 52, wherein the batch or the pharmaceutical composition is only validated for distribution when it contains less than 0.001% (w/w) of Compound A.

54. A process for preparing a batch of rivaroxaban (compound I) for pharmaceutical use, or a pharmaceutical composition comprising rivaroxaban, said process comprising determining the suitability for distribution of the batch of rivaroxaban, or the pharmaceutical composition comprising rivaroxaban, by a process comprising:
(a) producing a batch of rivaroxaban, or a pharmaceutical composition comprising rivaroxaban;
(b) measuring in a sample of the batch or the pharmaceutical composition the concentration of N,N′-bis[(5S)-2-oxo-3-[4-(3-oxomorpholin-4-yl)phenyl]-1,3-oxazolidin-5-yl}methyl]benzene-1,2-diamide (Compound B) using Compound B as a reference marker; and
(c) validating the batch or the pharmaceutical composition for distribution only if the sample of the batch or the pharmaceutical composition is free or substantially free of Compound B.

55. The process according to claim 54 wherein the batch or the pharmaceutical composition is only validated for distribution when it contains less than 0.15% (w/w) of Compound B.

56. The process according to claim 55, wherein the batch or the pharmaceutical composition is only validated for distribution when it contains less than 0.10% (w/w) of Compound B.

57. The process according to claim 56, wherein the batch or the pharmaceutical composition is only validated for distribution when it contains less than 0.05% (w/w) of Compound B.

58. The process according to claim 57, wherein the batch or the pharmaceutical composition is only validated for distribution when it contains less than 0.01% (w/w) of Compound B.

59. A process for preparing a batch of rivaroxaban (compound I) for pharmaceutical use, or a pharmaceutical composition comprising rivaroxaban, said process comprising determining the suitability for distribution of the batch of rivaroxaban, or the pharmaceutical composition comprising rivaroxaban, by a process comprising:
(a) producing a batch of rivaroxaban, or a pharmaceutical composition comprising rivaroxaban;
(b) measuring in a sample of the batch or the pharmaceutical composition the concentration of N,N-bis[{(5S)-2-oxo-3-[4-(3-oxomorpholin-4-yl)phenyl]-1,3-oxazolidin-5-yl}methyl]urea (Compound A) and the concentration of N,N′-bis[(5S)-2-oxo-3-[4-(3-oxomorpholin-4-yl)phenyl]-1,3-oxazolidin-5-yl}methyl]benzene-1,2-diamide (Compound B) using, respectively, Compound A and Compound B as reference markers; and
(c) validating the batch or the pharmaceutical composition for distribution only if the sample of the batch or the pharmaceutical composition is free or substantially free of Compound A and free or substantially free of Compound B.

60. The process according to claim 59, wherein the batch or the pharmaceutical composition is only validated for distribution when it contains less than 0.001% (w/w) of Compound A and less than 0.15% (w/w) of Compound B.

61. The process according to claim 60, wherein the batch or the pharmaceutical composition is only validated for distribution when it contains less than 0.10% (w/w) of Compound B.

62. The process according to claim 61, wherein the batch or the pharmaceutical composition is only validated for distribution when it contains less than 0.05% (w/w) of Compound B.

63. The process according to claim 62, wherein the batch or the pharmaceutical composition is only validated for distribution when it contains less than 0.01% (w/w) of Compound B.

64. Isolated N,N′-bis[{(5S)-2-oxo-3-[4-(3-oxomorpholin-4-yl)phenyl]-1,3-oxazolidin-5-yl}methyl]urea (Compound A) of formula,

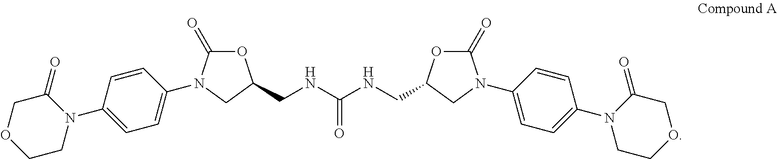 [SMILES: O=C(NC[C@H]1CN(c2ccc(N3CCOCC3=O)cc2)C(=O)O1)NC[C@H]1CN(c2ccc(N3CCOCC3=O)cc2)C(=O)O1]

65. Isolated N,N′-bis[(5S)-2-oxo-3-[4-(3-oxomorpholin-4-yl)phenyl]-1,3-oxazolidin-5-yl}methyl]benzene-1,2-diamide (Compound B) of formula,

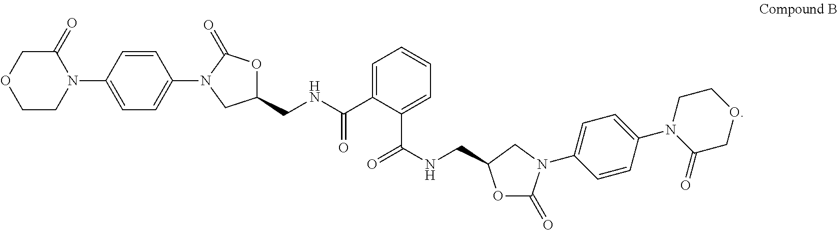 [SMILES: O=C(NC[C@H]1CN(c2ccc(N3CCOCC3=O)cc2)C(=O)O1)c1ccccc1C(=O)NC[C@H]1CN(c2ccc(N3CCOCC3=O)cc2)C(=O)O1]

66. A batch of rivaroxaban (compound I) for pharmaceutical use, or a pharmaceutical composition comprising rivaroxaban, prepared according to the process of claim 52.

67. A batch of rivaroxaban (compound I) for pharmaceutical use, or a pharmaceutical composition comprising rivaroxaban, prepared according to the process of claim 54.

68. A batch of rivaroxaban (compound I) for pharmaceutical use, or a pharmaceutical composition comprising rivaroxaban, prepared according to the process of claim 59.
